(19) United States
(12) Patent Application Publication
(10) Pub. No.: US 2023/0408477 A1
(43) Pub. Date: Dec. 21, 2023

(54) CROWDSOURCED INDOOR AIR QUALITY RATING SYSTEM

(71) Applicant: Air Rating, Inc., Canton, MI (US)

(72) Inventor: Deepak Mohan Raju, Canton, MI (US)

(73) Assignee: Air Rating, Inc., Canton, MI (US)

(21) Appl. No.: 18/359,210

(22) Filed: Jul. 26, 2023

(51) Int. Cl. G01N 33/00; F24F 11/58; B60H 1/00

(52) U.S. Cl. CPC G01N 33/0075; F24F 2110/50; B60H 1/008; F24F 11/58

(57) ABSTRACT

Embodiments relate to a system comprising a sensor module configured to detect and measure one or more air pollutants at a geographic location and generate air quality data, a cloud based application comprising a data processing module configured to receive, via a communication module, the air quality data and derive a first air quality index, a user device comprising an application program configured to receive, via the communication module, the first air quality index, the application program configured to suggest an alternate location having a second air quality index, and wherein the air quality data comprises one or more air quality parameters, the geographic location data corresponding to the one or more air quality parameters, and a time stamp; and wherein the system is configured to provide real-time air quality of the geographic location and the alternate location.

### FIELD OF INVENTION

The present invention relates to systems and methods for monitoring air quality, more specifically collecting and managing air quality data by crowdsourcing via a crowdsourcing platform and further leveraging the crowdsourced data.

### BACKGROUND

In this section prior art is cited:

“In one aspect, the present invention provides a sensor system that stays with a user whether indoors or outdoors, and senses air contaminants (analytes) or gasses in real time. When a user enters an area with poor air quality, or if a change in air quality is detected, the sensor warns the user by sending an alert to the user's smartphone and triggering a signal by the signal generator such as a vibration or audible warning at the device. In certain aspects, the system comprises crowdsourced data from nearby users, thus making the measurements and resulting data more robust.”[US20170023509A1 titled “Crowdsourced wearable sensor system”]

“A crowdsourced air quality monitoring system is disclosed in accordance with the various aspects and embodiments of the invention. The system monitors air quality in a given geographical area and provides information about the air quality for the given geographical area to a user.” [U.S. Ser. No. 11/112,395B2 titled “Networked air quality monitoring system”]

“While current building management/automation systems provide some degree of automated monitoring and controlling of a building's facilities and systems, such systems may still suffer from certain drawbacks, namely a lack of ability to sufficiently account for, among other things, occupant-driven data (e.g., input received from one or more occupants in a given space of the building, including occupant feedback and/or comfort information, as well as occupant-reported maintenance information) and subsequent on-the-fly updating of building automation based on occupant-driven data.” [U.S. Ser. No. 11/394,462B2 titled “Systems and methods for collecting, managing, and leveraging crowdsourced data”]

“The systems and methods described herein relate to the filtration and/or sensing of the air inhaled and/or exhaled by a user. The air that one inhales may contain numerous substances that are unpleasant or harmful. The systems and methods described herein may allow partial or complete filtration of these substances during inhalation, mitigating the harmful effects that these substances cause to a user's health. The systems and methods may allow users to obtain a customized filtration performance (such as a high level of filtration toward one or more harmful substances, a low level of filtration, or even a complete lack of filtration) based on the user's health concerns (such as allergies or residence in highly polluted areas) or personal preferences (such as athletic performance). The systems and methods may comprise replaceable filter modules that allow for the easy replacement of filters, such as when a filter suffers from reduced performance due to extensive use or when a user's filtration needs or desires change.” [US20200139165A1, titled “Systems and methods for respiratory health management”]

In scenarios like the coronavirus pandemic, people have experienced a lot of setbacks in their lives, and it is desirable to detect if the air quality is good or not for the people to choose to visit the place.

Therefore, there is a need to detect the air quality of a location and suggest to the user a remedial action when the detected air quality is poor.

### SUMMARY

The following presents a summary to provide a basic understanding of one or more embodiments described herein. This summary is not intended to identify key or critical elements or delineate any scope of the different embodiments and/or any scope of the claims. The sole purpose of the summary is to present some concepts in a simplified form as a prelude to the more detailed description presented herein.

According to an embodiment, it is a system comprising, a sensor module configured to detect and measure one or more air pollutants at a geographic location via one or more sensors and generate air quality data; a cloud based application comprising a data processing module configured to receive, via a communication module, the air quality data and derive a first air quality index; a user device comprising an application program configured to receive, via the communication module, the first air quality index; the application program configured to suggest an alternate location having a second air quality index, wherein the alternate location has better air quality than the geographic location; and wherein the air quality data comprises one or more air quality parameters, the geographic location corresponding to the one or more air quality parameters and a time stamp; and wherein the system is configured to provide real-time data of an air quality of the geographic location and the alternate location.

According to an embodiment of the system, the system is configured to generate the first air quality index by crowdsourcing via cloud computing.

According to an embodiment of the system, the system is configured to generate the second air quality index by crowdsourcing via cloud computing.

According to an embodiment of the system, the system is configured to generate an air quality index for a future period of time based on current air quality information and a history of air quality information.

According to an embodiment of the system, the system is configured to monitor air quality of an indoor space.

According to an embodiment of the system, the indoor space comprises one or more of an office space, a house, a restaurant, a café, a movie theater, a shopping mall, an office building, indoor spaces of a school, indoor spaces of a university, an indoor sports facility, an inside space of a public transportation vehicle, an inside space of a private transport vehicle, an indoor space of a hospital and healthcare facility, an underground parking lot, and an indoor concert venue.

According to an embodiment of the system, the system further comprises a control module configured to control a sterilization and circulation unit operable to remedy the air quality of the indoor space.

According to an embodiment of the system, the system further comprises a control module configured to control an Ultraviolet device that is configured to purify air in a vicinity of a user.

According to an embodiment of the system, the system is configured to monitor air quality of an outdoor space.

According to an embodiment of the system, the outdoor space comprises one or more of a park, a garden, a beach, a waterfront area, a playground, a sports field, an outdoor sports stadium, a hiking trail, a nature reserve, a public square, a plaza, an open-air market, an outdoor dining area, a patio, a campground, and a rooftop terrace.

According to an embodiment of the system, the sensor module comprises a sensor array with a plurality of air quality sensors, comprising a gas sensor, a particle sensor, and an environmental sensor.

According to an embodiment of the system, the one or more air quality parameters comprise CO2, volatile organic compounds, particulate matter, pathogens, temperature, humidity, and airflow.

According to an embodiment of the system, the sensor module comprises a global positioning system (GPS) sensor configured to generate the geographic location comprising longitude and latitude data.

According to an embodiment of the system, the sensor module is configured to monitor the air quality of the geographic location.

According to an embodiment of the system, the sensor module further comprises a power management module comprising a sleep mode to conserve battery life when the sensor module is not in use.

According to an embodiment of the system, the sensor module comprises a protective casing comprising shock-absorbing materials to protect the sensor module from accidental drops and impacts.

According to an embodiment of the system, the sensor module is portable.

According to an embodiment of the system, the sensor module comprises a location determination module comprising GPS.

According to an embodiment, wherein the air quality data is sent to the cloud based application by a smartphone which collects the air quality data from the sensor module using Bluetooth® connection, wherein a location data from the smartphone is also sent to the cloud based application.

According to an embodiment of the system, the application program generates an alert and notifies a user via one or more of an audio cue, a visual cue, a tactile cue, and a text message.

According to an embodiment of the system, the user device comprises a display configured to display the first air quality index.

According to an embodiment of the system, the data processing module comprises an artificial intelligence engine comprising a machine learning algorithm for identifying and classifying the one or more air pollutants.

According to an embodiment of the system, the machine learning algorithm is configured to analyze the air quality data collected from the sensor module, air quality monitoring stations and satellite imagery to identify patterns, to predict a pollution level and forecast air quality in the geographic location.

According to an embodiment of the system, the communication module is enabled for communication using one of a wired connection and a wireless connection.

According to an embodiment of the system, the communication module comprises one or more of a Wi-Fi, a Bluetooth®, and a cellular connectivity for data reception and data transmission.

According to an embodiment it is a method, comprising: sensing one or more air pollutants via a sensor module at a geographic location; generating air quality data; receiving the air quality data from the sensor module by an application program on a user device via a communication module; transmitting the air quality data to a server via the communication module; computing an air quality index from the air quality data via a data processing module; deriving an air quality rating corresponding to the air quality index; transmitting the air quality rating to the application program on the user device; and wherein the air quality data comprises one or more air quality parameters, the geographic location corresponding to the one or more air quality parameters and a time stamp; and wherein the method is configured to implement a crowdsourced sensor system for determining air quality.

According to an embodiment of the method, the method further comprises controlling an air purification system via a control module based on the air quality rating to improve the air quality.

According to an embodiment of the method, the air quality rating is transmitted to a second user device for real-time access of the air quality of the geographic location by a second user.

According to an embodiment of the method, the method further comprises a recommendation unit configured to recommend location along with the air quality to make informed decisions about an outdoor activity and a travel route based on the air quality rating.

According to an embodiment of the method, the air quality data is analyzed over time to identify patterns and trends enabling proactive measures to be taken to address potential air quality issues.

According to an embodiment of the method, the method further comprises a strategy to improve air quality comprising one or more of controlling a ventilation system, an air purifier, and implementing a source control measure to reduce emissions.

According to an embodiment of the method, the server comprises a web-based dashboard for visualizing and analyzing the air quality data from multiple geographic locations.

According to an embodiment of the method, the sensor module comprises a rechargeable battery and includes a fast charging feature for rapid recharging of the sensor module.

According to an embodiment of the method, the user device comprises a display module comprising a touch screen interface for user interaction.

According to an embodiment of the method, the sensor module comprises a protective case comprising a built-in charger for recharging the sensor device while it is stored in the protective case.

According to an embodiment of the method, the method is configured for predicting the air quality rating in a region comprising: collecting historical air quality data from one or more components of the sensor module; using a machine learning algorithm to identify a pattern and a trend in the historical air quality data; generating a predictive model for air quality for a future period; updating the predictive model with real-time air quality data; disseminating the air quality data for the future periodically to the public; and issuing alerts and recommendations based on a predicted air quality.

According to an embodiment of the method, the method is configured for monitoring air quality in a building comprising: installing one or more sensor modules in an indoor space; collecting the air quality data from the one or more sensor modules; transmitting the air quality data to a central server; analyzing the air quality data to identify potential sources of air pollution; implementing a strategy to improve air quality; and re-evaluating air quality over time to determine an effectiveness of the strategy.

According to an embodiment, it is a non-transitory computer-readable medium having stored thereon instructions executable by a computer system to perform operations comprising: receiving an air quality data on a server from an application program on a user device via a communication module; transmitting the air quality data to a server via the communication module; computing an air quality index from the air quality data via a data processing module; deriving an air quality rating corresponding to the air quality index; and transmitting the air quality rating to the application program on the user device; and wherein the air quality data comprises one or more air quality parameters, the geographic location corresponding to the one or more air quality parameters and a time stamp; and wherein the method is configured to implement a crowdsourced sensor system for determining air quality.

According to an embodiment it is an air quality measurement device comprising: a sensor module comprising an array of sensors to sense one or more air quality parameters and to generate air quality data; and a communication module to transmit the air quality data to a server; and wherein the array of sensors comprise one or more of a particulate matter sensor, a gas sensor, a nitrogen dioxide sensor, a carbon monoxide sensor, an ozone sensor, a volatile organic compound sensor, a biological sensor, a biochemical sensor; wherein the air quality data comprises carbon dioxide data, volatile organic compounds data, particulate matter data, humidity data, temperature data, location data, and a time stamp; wherein the air quality measurement device is configured to interface with a user device; and wherein the air quality measurement device is configured for measuring air quality at a geographic location.

According to an embodiment, it is an air quality measurement device comprising: a sensor module for detecting and measuring one or more air pollutants and generating air quality data; a location sensor for identifying a geographic location; a data processing unit for analyzing the air quality data and generating an air quality rating; a display module for displaying air quality rating to a user; a power management module for managing power usage in the sensor module; and a housing configured to house the air quality measurement device and to minimize damage of the air quality measurement device.

According to an embodiment of the air quality measurement device, the air quality measurement device is portable.

According to an embodiment of the air quality measurement device, the air quality measurement device comprises a compact housing for easy portability and shock absorption.

According to an embodiment of the air quality measurement device, the air quality measurement device comprises a protective case for safe storage and transportation.

According to an embodiment of the air quality measurement device, the air quality measurement device comprises a rechargeable battery configured for extended use.

According to an embodiment of the air quality measurement device, the air quality measurement device comprises a communication module for transmitting the air quality data to a remote device.

According to an embodiment, it is a method for measuring air quality comprising, detecting, and measuring one or more air pollutants using a sensor array; analyzing sensor data to determine an air quality index; presenting the air quality index to a user through a user interface; transmitting the air quality index to a remote device; and using the air quality index to decide on a control comprising a ventilation control and an air purification control of an indoor environment.

### BRIEF DESCRIPTION OF THE FIGURES

These and other aspects of the present invention will now be described in more detail, with reference to the appended drawings showing exemplary embodiments of the present invention, in which:

FIG. 1 shows a block diagram of the system configured for measuring air quality according to an embodiment.

FIG. 2 shows various components of the system configured for measuring air quality using crowdsourcing according to an embodiment.

FIG. 3A shows a structure of the neural network/machine learning model with a feedback loop according to an embodiment of the system.

FIG. 3B shows a structure of the neural network/machine learning model with reinforcement learning according to an embodiment of the system.

FIG. 3C shows an example block diagram for predicting air quality using a machine learning model according to an embodiment of the system.

FIG. 4 shows an example flow chart for predicting air quality using a machine learning model according to an embodiment of the system.

FIG. 5 shows an example display of air quality on a location map according to an embodiment of the system.

FIG. 6A shows an example of AQI indices and health concerns according to an embodiment of the system.

FIG. 6B shows an example display of quantified AQI using a user-friendly dial icon according to an embodiment of the system.

FIG. 7 shows a system suggesting alternate locations in indoor environments based on AQI ratings according to an embodiment.

FIG. 8 shows a block diagram of a method of determining the air quality according to an embodiment.

FIG. 9 shows the architecture of the system according to an embodiment.

FIG. 10 shows the Sensor module architecture according to an embodiment.

FIG. 11 shows Li-Ion Battery charger and Boost power supply circuit diagram according to an embodiment.

FIG. 12 shows a circuit diagram for ESP32 module according to an embodiment.

FIG. 13 shows USB-UART bridge circuit diagram for programming according to an embodiment.

FIG. 14 shows digital temperature humidity sensor circuit diagram according to an embodiment.

FIG. 15 shows a laser dust detection sensor circuit diagram according to an embodiment.

FIG. 16 shows a carbon dioxide and VOC sensor circuit diagram according to an embodiment.

FIG. 17 shows a VOC, temperature, and humidity sensor circuit diagram according to an embodiment.

FIG. 18 shows a smartphone application program interface according to an embodiment.

FIG. 19 shows a smartphone application menu interface according to an embodiment.

FIG. 20 shows a smartphone application displaying Air Quality parameters sensed by the sensor module according to an embodiment.

FIG. 21 shows a smartphone application for accessing Air Quality parameters according to an embodiment.

FIG. 22 shows a smartphone application displaying Air Quality parameters of a location of interest according to an embodiment.

### DETAILED DESCRIPTION

### Definitions and General Techniques

For simplicity and clarity of illustration, the figures illustrate the general manner of construction. The description and figures may omit the descriptions and details of well-known features and techniques to avoid unnecessarily obscuring the present disclosure. The figures exaggerate the dimensions of some of the elements relative to other elements to help improve understanding of embodiments of the present disclosure. The same reference numeral in different figures denotes the same element.

Although the detailed description herein contains many specifics for the purpose of illustration, a person of ordinary skill in the art will appreciate that many variations and alterations to the details are considered to be included herein.

Accordingly, the embodiments herein are without any loss of generality to, and without imposing limitations upon, any claims set forth. The terminology used herein is for the purpose of describing particular embodiments only and is not limiting. Unless defined otherwise, all technical and scientific terms used herein have the same meaning as commonly understood by one with ordinary skill in the art to which this disclosure belongs.

The following terms and phrases, unless otherwise indicated, shall be understood to have the following meanings.

As used herein, the articles “a” and “an” used herein refer to one or to more than one (i.e., to at least one) of the grammatical object of the article. By way of example, “an element” means one element or more than one element. Moreover, usage of articles “a” and “an” in the subject specification and annexed drawings construe to mean “one or more” unless specified otherwise or clear from context to mean a singular form.

As used herein, the terms “example” and/or “exemplary” mean serving as an example, instance, or illustration. For the avoidance of doubt, such examples do not limit the herein described subject matter. In addition, any aspect or design described herein as an “example” and/or “exemplary” is not necessarily preferred or advantageous over other aspects or designs, nor does it preclude equivalent exemplary structures and techniques known to those of ordinary skill in the art.

As used herein, the terms “first,” “second,” “third,” and the like in the description and in the claims, if any, distinguish between similar elements and do not necessarily describe a particular sequence or chronological order. The terms are interchangeable under appropriate circumstances such that the embodiments herein are, for example, capable of operation in sequences other than those illustrated or otherwise described herein. Furthermore, the terms “include,” “have,” and any variations thereof, cover a non-exclusive inclusion such that a process, method, system, article, device, or apparatus that comprises a list of elements is not necessarily limiting to those elements, but may include other elements not expressly listed or inherent to such process, method, system, article, device, or apparatus.

As used herein, the terms “left,” “right,” “front,” “back,” “top,” “bottom,” “over,” “under” and the like in the description and in the claims, if any, are for descriptive purposes and not necessarily for describing permanent relative positions. The terms so used are interchangeable under appropriate circumstances such that the embodiments of the apparatus, methods, and/or articles of manufacture described herein are, for example, capable of operation in other orientations than those illustrated or otherwise described herein.

No element act, or instruction used herein is critical or essential unless explicitly described as such. Furthermore, the term “set” includes items (e.g., related items, unrelated items, a combination of related items and unrelated items, etc.) and may be interchangeable with “one or more”. Where only one item is intended, the term “one” or similar language is used. Also, the terms “has,” “have,” “having,” or the like are open-ended terms. Further, the phrase “based on” means “based, at least in part, on” unless explicitly stated otherwise.

As used herein, the terms “couple,” “coupled,” “couples,” “coupling,” and the like refer to connecting two or more elements mechanically, electrically, and/or otherwise. Two or more electrical elements may be electrically coupled together, but not mechanically or otherwise coupled together. Coupling may be for any length of time, e.g., permanent, or semi-permanent or only for an instant. “Electrical coupling” includes electrical coupling of all types. The absence of the word “removably,” “removable,” and the like, near the word “coupled” and the like does not mean that the coupling, etc. in question is or is not removable.

As used herein, the terms “system,” “device,” “unit,” and/or “module” refer to a different component, component portion, or component of the various levels of the order. However, other expressions that achieve the same purpose may replace the terms.

As used herein, the term “or” means an inclusive “or” rather than an exclusive “or.” That is, unless specified otherwise, or clear from context, “X employs A or B” means any of the natural inclusive permutations. That is, if X employs A; X employs B; or X employs both A and B, then “X employs A or B” is satisfied under any of the foregoing instances.

As used herein, two or more elements or modules are “integral” or “integrated” if they operate functionally together. Two or more elements are “non-integral” if each element can operate functionally independently.

As used herein, the term “real-time” refers to operations conducted as soon as practically possible upon occurrence of a triggering event. A triggering event can include receipt of data necessary to execute a task or to otherwise process information. Because of delays inherent in transmission and/or in computing speeds, the term “real-time” encompasses operations that occur in “near” real-time or somewhat delayed from a triggering event. In a number of embodiments, “real-time” can mean real-time less a time delay for processing (e.g., determining) and/or transmitting data. The particular time delay can vary depending on the type and/or amount of the data, the processing speeds of the hardware, the transmission capability of the communication hardware, the transmission distance, etc. However, in many embodiments, the time delay can be less than approximately one second, two seconds, five seconds, or ten seconds.

The present disclosure may be embodied in other specific forms without departing from its spirit or characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

Unless otherwise defined herein, scientific and technical terms used in connection with the present disclosure shall have meanings that are commonly understood by those of ordinary skill in the art. Further, unless otherwise required by context, singular terms shall include pluralities and plural terms shall include the singular.

As used herein, the term “approximately” can mean within a specified or unspecified range of the specified or unspecified stated value. In some embodiments, “approximately” can mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Other specific forms may embody the present invention without departing from its spirit or characteristics. The described embodiments are in all respects illustrative and not restrictive. Therefore, the appended claims rather than the description herein indicate the scope of the invention. All variations which come within the meaning and range of equivalency of the claims are within their scope.

As used herein, the term “component” broadly construes hardware, firmware, and/or a combination of hardware, firmware, and software.

Digital electronic circuitry, or in computer software, firmware, or hardware, including the structures disclosed in this specification and their structural equivalents, or in combinations of one or more of them may realize the implementations and all of the functional operations described in this specification. Implementations may be as one or more computer program products i.e., one or more modules of computer program instructions encoded on a computer-readable medium for execution by, or to control the operation of, data processing apparatus. The computer-readable medium may be a machine-readable storage device, a machine-readable storage substrate, a memory device, a composition of matter affecting a machine-readable propagated signal, or a combination of one or more of them. The term “computing system” encompasses all apparatus, devices, and machines for processing data, including by way of example, a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal (e.g., a machine-generated electrical, optical, or electromagnetic signal) that encodes information for transmission to a suitable receiver apparatus.

The actual specialized control hardware or software code used to implement these systems and/or methods is not limiting to the implementations. Thus, any software and any hardware can implement the systems and/or methods based on the description herein without reference to specific software code.

A computer program (also known as a program, software, software application, script, or code) is written in any appropriate form of programming language, including compiled or interpreted languages. Any appropriate form, including a standalone program or a module, component, subroutine, or other unit suitable for use in a computing environment may deploy it. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may execute on one computer or on multiple computers that are located at one site or distributed across multiple sites and interconnected by a communication network.

One or more programmable processors, executing one or more computer programs to perform functions by operating on input data and generating output, perform the processes and logic flows described in this specification. The processes and logic flows may also be performed by, and apparatus may also be implemented as, special purpose logic circuitry, for example, without limitation, a Field Programmable Gate Array (FPGA), an Application Specific Integrated Circuit (ASIC), Application Specific Standard Products (ASSPs), System-On-a-Chip (SOC) systems, Complex Programmable Logic Devices (CPLDs), etc.

Processors suitable for the execution of a computer program include, by way of example, both general and special purpose microprocessors, and any one or more processors of any appropriate kind of digital computer. A processor will receive instructions and data from a read-only memory or a random-access memory or both. Elements of a computer can include a processor for performing instructions and one or more memory devices for storing instructions and data. A computer will also include, or is operatively coupled to receive data, transfer data or both, to/from one or more mass storage devices for storing data e.g., magnetic disks, magneto optical disks, optical disks, or solid-state disks. However, a computer need not have such devices. Moreover, another device, e.g., a mobile telephone, a personal digital assistant (PDA), a mobile audio player, a Global Positioning System (GPS) receiver, etc. may embed a computer. Computer-readable media suitable for storing computer program instructions and data include all forms of non-volatile memory, media and memory devices, including, by way of example, semiconductor memory devices (e.g., Erasable Programmable Read-Only Memory (EPROM), Electronically Erasable Programmable Read-Only Memory (EEPROM), and flash memory devices), magnetic disks (e.g., internal hard disks or removable disks), magneto optical disks (e.g. Compact Disc Read-Only Memory (CD ROM) disks, Digital Versatile Disk-Read-Only Memory (DVD-ROM) disks) and solid-state disks. Special purpose logic circuitry may supplement or incorporate the processor and the memory.

To provide for interaction with a user, a computer may have a display device, e.g., a Cathode Ray Tube (CRT) or Liquid Crystal Display (LCD) monitor, for displaying information to the user, and a keyboard and a pointing device, e.g., a mouse or a trackball, by which the user may provide input to the computer. Other kinds of devices provide for interaction with a user as well. For example, feedback to the user may be any appropriate form of sensory feedback, e.g., visual feedback, auditory feedback, or tactile feedback; and a computer may receive input from the user in any appropriate form, including acoustic, speech, or tactile input.

A computing system that includes a back-end component, e.g., a data server, or that includes a middleware component, e.g., an application server, or that includes a front-end component, e.g., a client computer having a graphical user interface or a Web browser through which a user may interact with an implementation, or any appropriate combination of one or more such back-end, middleware, or front-end components, may realize implementations described herein. Any appropriate form or medium of digital data communication, e.g., a communication network may interconnect the components of the system. Examples of communication networks include a Local Area Network (LAN) and a Wide Area Network (WAN), e.g., Intranet and Internet.

The computing system may include clients and servers. A client and server are remote from each other and typically interact through a communication network. The relationship of the client and server arises by virtue of computer programs running on the respective computers and having a client-server relationship with each other.

Embodiments of the present invention may comprise or utilize a special purpose or general purpose computer including computer hardware. Embodiments within the scope of the present invention may also include physical and other computer-readable media for carrying or storing computer-executable instructions and/or data structures. Such computer-readable media can be any media accessible by a general purpose or special purpose computer system. Computer-readable media that store computer-executable instructions are physical storage media. Computer-readable media that carry computer-executable instructions are transmission media. Thus, by way of example and not limitation, embodiments of the invention can comprise at least two distinct kinds of computer-readable media: physical computer-readable storage media and transmission computer-readable media.

Although the present embodiments described herein are with reference to specific example embodiments it will be evident that various modifications and changes may be made to these embodiments without departing from the broader spirit and scope of the various embodiments. For example, hardware circuitry (e.g., Complementary Metal Oxide Semiconductor (CMOS) based logic circuitry), firmware, software (e.g., embodied in anon-transitory machine-readable medium), or any combination of hardware, firmware, and software may enable and operate the various devices, units, and modules described herein. For example, transistors, logic gates, and electrical circuits (e.g., Application Specific Integrated Circuit (ASIC) and/or Digital Signal Processor (DSP) circuit) may embody the various electrical structures and methods.

In addition, a non-transitory machine-readable medium and/or a system may embody the various operations, processes, and methods disclosed herein. Accordingly, the specification and drawings are illustrative rather than restrictive.

Physical computer-readable storage media includes RAM, ROM, EEPROM, CD-ROM or other optical disk storage (such as CDs, DVDs, etc.), magnetic disk storage or other magnetic storage devices, solid-state disks or any other medium. They store desired program code in the form of computer-executable instructions or data structures which can be accessed by a general purpose or special purpose computer.

Further, upon reaching various computer system components, program code in the form of computer-executable instructions or data structures can be transferred automatically from transmission computer-readable media to physical computer-readable storage media (or vice versa). For example, computer-executable instructions or data structures received over a network or data link can be buffered in RAM within a Network Interface Module (NICK), and then eventually transferred to computer system RAM and/or to less volatile computer-readable physical storage media at a computer system. Thus, computer system components that also (or even primarily) utilize transmission media may include computer-readable physical storage media.

Computer-executable instructions comprise, for example, instructions and data which cause a general purpose computer, special purpose computer, or special purpose processing device to perform a certain function or group of functions. The computer-executable instructions may be, for example, binary, intermediate format instructions such as assembly language, or even source code. Although the subject matter herein described is in a language specific to structural features and/or methodological acts, the described features or acts described do not limit the subject matter defined in the claims. Rather, the herein described features and acts are example forms of implementing the claims.

While this specification contains many specifics, these do not construe as limitations on the scope of the disclosure or of the claims, but as descriptions of features specific to particular implementations. A single implementation may implement certain features described in this specification in the context of separate implementations. Conversely, multiple implementations separately or in any suitable sub-combination may implement various features described herein in the context of a single implementation. Moreover, although features described herein as acting in certain combinations and even initially claimed as such, one or more features from a claimed combination may in some cases be excised from the combination, and the claimed combination may be directed to a sub-combination or variation of a sub-combination.

Similarly, while operations depicted herein in the drawings in a particular order to achieve desired results, this should not be understood as requiring that such operations be performed in the particular order shown or in sequential order or that all illustrated operations be performed, to achieve desirable results. In certain circumstances, multitasking and parallel processing may be advantageous. Moreover, the separation of various system components in the implementations should not be understood as requiring such separation in all implementations, and it should be understood that the described program components and systems may be integrated together in a single software product or packaged into multiple software products.

Even though particular combinations of features are recited in the claims and/or disclosed in the specification, these combinations are not intended to limit the disclosure of possible implementations. Other implementations are within the scope of the claims. For example, the actions recited in the claims may be performed in a different order and still achieve desirable results. In fact, many of these features may be combined in ways not specifically recited in the claims and/or disclosed in the specification. Although each dependent claim may directly depend on only one claim, the disclosure of possible implementations includes each dependent claim in combination with every other claim in the claim set.

Further, a computer system including one or more processors and computer-readable media such as computer memory may practice the methods. In particular, one or more processors execute computer-executable instructions, stored in the computer memory, to perform various functions such as the acts recited in the embodiments.

Those skilled in the art will appreciate that the invention may be practiced in network computing environments with many types of computer system configurations including personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, etc. Distributed system environments where local and remote computer systems, which are linked (either by hardwired data links, wireless data links, or by a combination of hardwired and wireless data links) through a network, both perform tasks may also practice the invention. In a distributed system environment, program modules may be located in both local and remote memory storage devices.

As used herein, the term “IoT” stands for Internet of Things which describes the network of physical objects “things” or objects embedded with sensors, software, and other technologies for the purpose of connecting and exchanging data with other devices and systems over the internet.

As used herein “Machine learning” refers to algorithms that give a computer the ability to learn without explicit programming, including algorithms that learn from and make predictions about data. Machine learning techniques include, but are not limited to, support vector machine, artificial neural network (ANN) (also referred to herein as a “neural net”), deep learning neural network, logistic regression, discriminant analysis, random forest, linear regression, rules-based machine learning, Naive Bayes, nearest neighbor, decision tree, decision tree learning, and hidden Markov, etc. For the purposes of clarity, part of a machine learning process can use algorithms such as linear regression or logistic regression. However, using linear regression or another algorithm as part of a machine learning process is distinct from performing a statistical analysis such as regression with a spreadsheet program. The machine learning process can continually learn and adjust the classifier as new data becomes available and does not rely on explicit or rules-based programming. The ANN may be featured with a feedback loop to adjust the system output dynamically as it learns from the new data as it becomes available. In machine learning, backpropagation and feedback loops are used to train the Artificial Intelligence/Machine Learning (AI/ML) model improving the model's accuracy and performance over time.

Statistical modeling relies on finding relationships between variables (e.g., mathematical equations) to predict an outcome.

As used herein, the term “Data mining” is a process used to turn raw data into useful information.

As used herein, the term “Data acquisition” is the process of sampling signals that measure real world physical conditions and converting the resulting samples into digital numeric values that a computer manipulates. Data acquisition systems typically convert analog waveforms into digital values for processing. The components of data acquisition systems include sensors to convert physical parameters to electrical signals, signal conditioning circuitry to convert sensor signals into a form that can be converted to digital values, and analog-to-digital converters to convert conditioned sensor signals to digital values. Stand-alone data acquisition systems are often called data loggers.

As used herein, the term “Dashboard” is a type of interface that visualizes particular Key Performance Indicators (KPIs) for a specific goal or process. It is based on data visualization and infographics.

As used herein, a “Database” is a collection of organized information so that it can be easily accessed, managed, and updated. Computer databases typically contain aggregations of data records or files.

As used herein, the term “Data set” (or “Dataset”) is a collection of data. In the case of tabular data, a data set corresponds to one or more database tables, where every column of a table represents a particular variable, and each row corresponds to a given record of the data set in question. The data set lists values for each of the variables, such as height and weight of an object, for each member of the data set. Each value is known as a datum. Data sets can also consist of a collection of documents or files.

As used herein, a “Sensor” is a device that measures physical input from its environment and converts it into data that is interpretable by either a human or a machine. Most sensors are electronic, which presents electronic data, but some are simpler, such as a glass thermometer, which presents visual data.

The term “communication module” or “communication system” as used herein refers to a system which enables the information exchange between two points. The process of transmission and reception of information is called communication. The elements of communication include but are not limited to a transmitter of information, channel or medium of communication and a receiver of information.

The term “connection” as used herein refers to a communication link. It refers to a communication channel that connects two or more devices for the purpose of data transmission. It may refer to a physical transmission medium such as a wire, or to a logical connection over a multiplexed medium such as a radio channel in telecommunications and computer networks. A channel is used for the information transfer of, for example, a digital bit stream, from one or several senders to one or several receivers. A channel has a certain capacity for transmitting information, often measured by its bandwidth in Hertz (Hz) or its data rate in bits per second.

The term “communication” as used herein refers to the transmission of information and/or data from one point to another. Communication may be by means of electromagnetic waves. Communication is also a flow of information from one point, known as the source, to another, the receiver. Communication comprises one of the following: transmitting data, instructions, information or a combination of data, instructions, and information. Communication happens between any two communication systems or communicating units.

Further, the communication apparatus is configured on a computer with the communication function and is connected for bidirectional communication by a communication line through a radio station and a communication network such as a public telephone network or by satellite communication through a communication satellite. The communication apparatus is adapted to communicate, through the communication network, with communication terminals.

The term “bidirectional communication” as used herein refers to an exchange of data between two components. In an example, the first component can be a user device and the second component can be an air sensing device.

The term “alert” or “alert signal” refers to a communication to attract attention. An alert may include visual, tactile, audible alert, and a combination of these alerts to warn drivers or occupants. These alerts allow receivers, such as drivers or occupants, the ability to react and respond quickly.

The term “in communication with” as used herein, refers to any coupling, connection, or interaction using signals to exchange information, message, instruction, command, and/or data, using any system, hardware, software, protocol, or format regardless of whether the exchange occurs wirelessly or over a wired connection.

As used herein, the term “network” refers to one or more data links that enable the transport of electronic data between computer systems and/or modules and/or other electronic devices. When a network or another communications connection (either hardwired, wireless, or a combination of hardwired or wireless) transfers or provides information to a computer, the computer properly views the connection as a transmission medium. A general purpose or special purpose computer access transmission media that can include a network and/or data links which carry desired program code in the form of computer-executable instructions or data structures. The scope of computer-readable media includes combinations of the above, that enable the transport of electronic data between computer systems and/or modules and/or other electronic devices. The network may include one or more networks or communication systems, such as the Internet, the telephone system, satellite networks, cable television networks, and various other private and public networks. In addition, the connections may include wired connections (such as wires, cables, fiber optic lines, etc.), wireless connections, or combinations thereof. Furthermore, although not shown, other computers, systems, devices, and networks may also be connected to the network. Network refers to any set of devices or subsystems connected by links joining (directly or indirectly) a set of terminal nodes sharing resources located on or provided by network nodes. The computers use common communication protocols over digital interconnections to communicate with each other. For example, subsystems may comprise the cloud. Cloud refers to servers that are accessed over the Internet, and the software and databases that run on those servers.

The terms “non-transitory computer-readable medium” and “computer-readable medium” include a single medium or multiple media such as a centralized or distributed database, and/or associated caches and servers that store one or more sets of instructions. Further, the terms “non-transitory computer-readable medium” and “computer-readable medium” include any tangible medium that is capable of storing, encoding, or carrying a set of instructions for execution by a processor that, for example, when executed, cause a system to perform any one or more of the methods or operations disclosed herein. As used herein, the term “computer readable medium” is expressly defined to include any type of computer readable storage device and/or storage disk and to exclude propagating signals.

The methods and techniques of the present disclosure are generally performed according to conventional methods well known in the art and as described in various general and more specific references that are cited and discussed throughout the present specification unless otherwise indicated. The nomenclatures used in connection with, and the procedures and techniques of, the embodiments herein, and other related fields described herein are those well-known and commonly used in the art.

The term “air quality” as used herein refers to the overall condition of the air in a particular area, indicating the presence of pollutants and the degree of cleanliness. It is a measure of the presence of pollutants, such as particulate matter (PM), gases, and other harmful substances, in the atmosphere.

The term “air pollutant” as used herein refers to substances or particles that are present in the air and can have harmful effects on human health, animals, and the environment. These pollutants can be either natural or human-made and can be in the form of gases, particles, or biological molecules. Examples of air pollutants include carbon monoxide (CO), nitrogen oxides (NOx), sulfur dioxide (SO2), ozone (O3), particulate matter (PM), volatile organic compounds (VOCs), and biological pollutants like pollen and mold spores.

The term “air quality data” as used herein refers to the measurements and readings of one or more pollutants and other parameters in the air, such as particulate matter (PM), nitrogen dioxide (NO2), ozone (O3), sulfur dioxide (SO2), carbon monoxide (CO), volatile organic compounds (VOCs), temperature, humidity, and air pressure.

The term “Air Quality Index (AQI)” as used herein refers to a standardized measurement system used to assess and communicate the quality of air. It provides a numerical value or index to indicate the relative level of pollution and its potential health effects. The AQI considers various pollutants, such as particulate matter (PM2.5 and PM10), ozone (O3), nitrogen dioxide (NO2), sulfur dioxide (SO2), and carbon monoxide (CO), and converts their concentrations into a single value that is easy to understand. The AQI scale typically ranges from 0 to 500, with higher values indicating poorer air quality and increased health risks. The index may often be accompanied by descriptive categories, such as “Good,” “Moderate,” “Unhealthy,” “Very Unhealthy,” and “Hazardous,” to provide further information about the air quality conditions.

The term “air quality information” as used herein refers to data and details about the current state of the air quality in a specific location. It includes measurements and analysis of various pollutants present in the atmosphere, such as particulate matter, gases, and other harmful substances. This information is typically collected through air quality monitoring stations or sensors in different areas. The data collected from these stations or sensors is used to assess the level of pollution, determine the air quality index (AQI), and provide updates and alerts to the public regarding the quality of the air they are breathing.

The term “Indoor Air Quality (IAQ) ratings” as used herein provides an assessment of the cleanliness and healthiness of the air within enclosed spaces. Ratings typically range from excellent to hazardous, indicating the level of pollutants present and their potential impact on occupants' health. Excellent and good ratings reflect clean and healthy air, while fair indicates moderate pollution levels. Poor and hazardous ratings signify high pollution levels requiring immediate action. Maintaining good IAQ involves monitoring, ventilation, filtration, and minimizing pollutant sources to ensure a healthy indoor environment.

The term “crowdsourcing” as used herein refers to the practice of obtaining ideas, information, services, or solutions by soliciting contributions from a large group of people, typically through an open call or online platform. It involves harnessing the collective intelligence, skills, and resources of a diverse crowd to accomplish tasks or solve problems. Examples of crowdsourcing include online platforms where users contribute content, such as Wikipedia, or platforms where individuals collectively fund projects, like crowdfunding platforms.

With the increase in miniaturization, portable and wearable electronic products are a growing trend. As such, sophisticated air quality monitoring instruments are increasingly within reach of individual users. Such instruments, however, have a limited range and can only measure conditions in the immediate vicinity of the instrument. Air quality is of increasing concern for individuals. Numerous studies have been published linking air quality with short and long term health problems. For example: The World Health Organization (WHO) estimates that some 80% of outdoor air pollution-related premature deaths were due to ischemic heart disease and strokes, while 14% of deaths were due to chronic obstructive pulmonary disease or acute lower respiratory infections, and 6% of deaths were due to lung cancer.

Ambient outdoor air pollution in both cities and rural areas was estimated to cause 3.7 million premature deaths worldwide per year in 2012. This mortality is due to exposure to small particulate matter of 10 microns or less in diameter, PM10, which causes cardiovascular and respiratory disease, and cancers. The 2005 “WHO Air quality guidelines” offer global guidance on thresholds and limits for key air pollutants that pose health risks. The Guidelines indicate that by reducing particulate matter PM10 pollution from 70 to 20 micrograms per cubic meter μg/m3, air pollution-related deaths can be reduced by around 15%.

The Guidelines apply worldwide and are based on expert evaluation of current scientific evidence in all WHO regions for: particulate matter (PM); ozone (O3); nitrogen dioxide (NO2); and sulfur dioxide (SO2).

In addition to the above, a number of other parameters affecting health are also cited by other sources like the Centers for Disease Control CDC. These include Carbon Monoxide, Lead, Nitrogen Oxide, Acrolein, Asbestos, Benzene, Carbon Disulfide, Creosote, Fuel oils like Kerosene, Polycyclic Aromatic Hydrocarbons, Synthetic Vitreous Fibers, Total Petroleum Hydrocarbons. Synthetic vitreous fibers are a group of fibrous inorganic materials that contain aluminum or calcium silicates and other trace oxides and metals, and are made from rock, slag, clay, or glass. Various other volatile organic compounds like Formaldehyde are also of interest since products manufactured with these are present or sold in some countries. Exposure to air pollutants can lead to a variety of health problems, including respiratory diseases, heart disease, cancer, and neurological damage. Therefore, there is a need for a system that monitors air quality in a given geographical area and provides information about air quality for the given geographical area to a user.

In the current invention, air quality data may be collected using portable air quality devices. However, air quality data may be collected through various methods, such as air quality monitoring stations, or satellite observations. This data may be used to assess the quality of the air, identify sources of pollution, evaluate the effectiveness of air pollution control strategies, and make informed decisions related to public health and the environment. Data processing is the set of operations performed on data to transform it into a more useful form. It involves various tasks such as collection, manipulation, storage, retrieval, and analysis of data. Data processing can be performed manually or using computer software programs and algorithms. The goal of data processing is to extract meaningful information from the data, identify patterns and trends, and provide insights that can be used to make informed decisions. The process may involve cleaning and filtering the data to remove errors, duplicates, or irrelevant information, transforming the data into a more structured format, and analyzing the data using statistical or machine learning algorithms to extract meaningful insights.

Most humans spend 90% of their time indoors, making it crucial to maintain good indoor air quality. Indoor air quality has been linked to numerous health conditions such as respiratory illnesses and asthma. In addition, poor indoor air quality is responsible for an increase in the spread of airborne infections such as the Coronavirus. Also, poor indoor air quality has a negative impact on cognitive functioning, hindering productivity at work or school. For these reasons, good indoor air quality is vital to health and happiness. There may be several agencies monitoring the outdoor air quality and pollution of the environment using air quality monitoring stations. However, there is very limited or no monitoring of the air quality indoors including public and personal spaces.

Current invention uses crowdsourced air quality information to make available air quality ratings of indoor spaces so that people can access/validate the hygiene of locations that they would like to live, visit, or work. The air quality ratings are made available to the public on a web portal that can be accessed by the public online on a smartphone and/or on a computer.

This invention improves over previously known processes by simultaneously utilizing crowdsourced data, numerous air sensors (sensing carbon dioxide, temperature, humidity, volatile organic compounds, and particulate matter), and cloud computing to make available air quality ratings to the public of indoor spaces they would like to visit, live, or work at. Using these air sensors, the air quality of the given indoor space can be measured. Air sensors measure carbon dioxide, temperature, humidity, volatile organic compounds, pathogens, and particulate matter which are then used to determine air quality.

In an embodiment, the system comprises an air quality sensor as a first component, a smartphone application as a second component, and a cloud native application as a third component. Smartphone application is called as AirNearMe® application and the cloud native application is an Amazon Web Services (AWS® cloud driven) AirNearMe® web portal. The first component, the sensor, measures air quality parameters of the indoor air such as carbon dioxide, volatile organic compounds, particulate matter, humidity, and temperature. This data is received by a smartphone application, AirNearMe®, (the second component) via a Bluetooth® connection which is processed and displayed as individual parameters and as good, moderate, or bad based on an IAQ (indoor air quality) ratings. An IAQ rating is an internationally and U.S recognized standard to measure indoor air quality. This data (as displayed on the AirNearMe® app) along with the GPS coordinates (of the location the data is captured) as transmitted to the AWS® cloud by the AirNearMe® application. Using the GPS information, the AirNearMe® app matches the indoor air quality data to the location (for example an address, location coordinates, and a location name) it comes from. The AirNearMe® web portal makes the indoor air quality data available to the public for viewing on the web or on the AirNearMe® app on a smartphone.

In an embodiment, the public gathering of air quality data is obtained with an air sensor. A smartphone application and cloud infrastructure analyze the gathered data, rate, and display the air quality of indoor public spaces globally to the general public. Through a combination of these three features, obtaining, analyzing, displaying, the invention can gather crowdsourced air quality data from the public, send this data to a smartphone application which rates the air quality and uploads it to the cloud so that the public is able to view it. This allows for the crowdsourcing of the air quality of indoor spaces and makes this information available to the public (by viewing the information online). In an aspect of the invention, people can be aware of the air quality in places they visit, live, or work through the application or using the system. The places they live, visit, or work could include restaurants, auditoriums, hotels, apartments, office buildings, classrooms, etc.

This invention comprises a process where individuals of the public gather indoor air quality data using technology which comprises air sensor, smartphone application, and cloud infrastructure, to generate air quality ratings of indoor public locations and make these ratings available to the public for their respective uses.

In an embodiment, the process uses individuals to provide air quality for the air quality rating of a geographic location. People spend 90% of time indoors whether it is at home, work, or at schools. Because of this, it is crucial to be aware of the state of the indoor air quality in the indoor spaces. Despite this, there is very little information available on the air quality indoors. Poor indoor air quality has numerous adverse impacts on health by causing health problems like asthma, allergies, cardiovascular diseases, respiratory illnesses, and many more. Also, in conditions with poor indoor air quality, the transmission of pathogens substantially increases the contraction of airborne illnesses such as the Coronavirus which was recently experienced where millions of people died.

In an embodiment, the system will allow the public to be aware of the indoor air quality of the locations they frequent or wish to visit in their everyday lives. Because of this, people would be able to make conscious decisions when choosing the places they would like to live, visit, or work based on the indoor air quality of the location. In an embodiment, awareness of indoor air quality may drive efforts to improve indoor air quality around the world. Efforts to improve the air quality indoors greatly improves the health of the public by reducing the spread of transmissible diseases and reducing respiratory and cardiovascular illnesses.

In an embodiment, air was analyzed using the sensor at various geographic locations. At these locations, the system has sensed the air quality of the place (air sensor has sensed carbon dioxide, temperature, humidity, particulate matter, and volatile organic compound levels at the location) and transmitted this air quality data of the geographic locations to the smartphone where the air quality data was displayed on the application and the air quality at the geographic locations was rated. Once the air quality of the location was rated, the smartphone application uploaded the rating to the cloud. In an embodiment, the application and web portal are presented in a user-friendly manner. In an embodiment, the air sensor module is calibrated and tested to validate accuracy of readings.

Presently the indoor air quality of locations for the public are not available. Much of the emphasis on air quality has largely been focused on outdoor air using air quality monitoring stations that are fixed at the location and are huge. There are many air sensors sold by companies which solely display the air quality in the location of which the sensor has been purchased for.

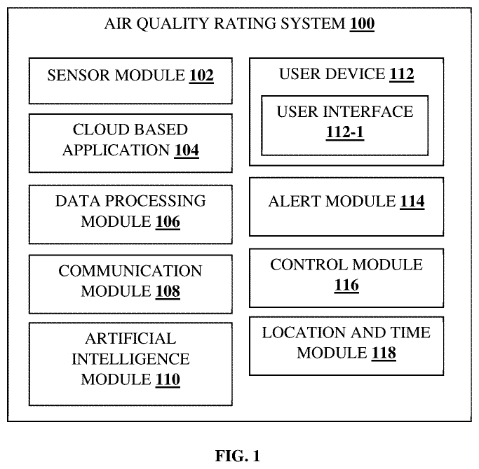

FIG. 1 shows a block diagram of the air quality rating system according to an embodiment. Air quality rating system 100 comprises a sensor module 102, a cloud-based application 104, a data processing module 106, a communication module 108, an artificial intelligence module 110, a user device 112 comprising a user interface 112-1, an alert module 114, a control module 116 and a location and time module 118.

Sensor module 102: A sensor module 102 may comprise several components that work together to detect and measure specific physical or environmental parameters. Illustrated herein are components of the sensor module:

Sensor Element: This is the core component of the sensor module responsible for detecting and converting the physical or environmental stimulus into an electrical signal. Sensor elements may be used to detect one or more air pollutants and include particulate matter sensors, gas-sensitive elements, nitrogen dioxide sensors, carbon monoxide sensors, ozone sensors, and volatile organic compound sensors. Sensor elements may also detect pathogens and may further comprise biological or biochemical sensors, such as biosensors or immunosensors. These sensors utilize specific biological recognition elements, such as antibodies or enzymes, to detect the presence of pathogens by binding to their unique molecular components or antigens. For example, Polymerase Chain Reaction (PCR) may detect various pathogens, including bacteria, viruses, and fungi; Anti-Influenza Antibody may detect influenza viruses; Lactate Dehydrogenase (LDH) may detect Lactobacillus species, commonly used as an indicator of microbial contamination in food or clinical samples; Beta-lactamase Pathogen may be used to detect antibiotic-resistant bacteria, such as methicillin-resistant Staphylococcus aureus (MRSA); Reverse Transcriptase (RT) may detect RNA viruses, such as HIV and hepatitis C virus (HCV). The sensor module may comprise a strip or a rod where antibodies can be integrated into strip-based formats to enable easy and portable pathogen detection. These assays utilize specific antibodies or other recognition elements immobilized on a strip or a rod, allowing for the capture and detection of the target pathogen. For example, streptavidin and anti-Salmonella detection. These strips or rods need to be replaced once used.

Signal Conditioning Circuitry: The electrical signal generated by the sensor element often requires conditioning to enhance its accuracy, sensitivity, and compatibility with the measurement system. This circuitry may include amplifiers, filters, analog-to-digital converters (ADCs), and other components to process and prepare the signal for further analysis.

Interface Electronics: The sensor module may include interface electronics to communicate with external devices or systems. This can include digital interfaces such as I2C (Inter-Integrated Circuit), SPI (Serial Peripheral Interface), UART (Universal Asynchronous Receiver-Transmitter), or analog interfaces for direct voltage or current output.

Power Supply Circuitry: The sensor module may have power supply circuitry to provide the necessary voltage or current to operate the sensor element and the associated electronics. This can include voltage regulators, power management circuits, or conditioning circuits to ensure stable and reliable power supply.

Packaging and Enclosure: The sensor module is typically housed within a protective package or enclosure to safeguard the internal components from environmental factors, such as moisture, dust, falls, and/or mechanical stress. The packaging also provides mechanical support and facilitates easy integration into various applications.

Connectors or Pins: The sensor module may feature connectors or pins for electrical connections, allowing it to be easily connected to external devices or integrated into larger systems.

Control and Calibration Components: Sensor modules may include control elements for configuring sensor settings and performing calibration procedures. These components enable customization, calibration, or adjustment of the sensor's performance to suit specific application requirements.

In an embodiment, specific components and configuration of sensors and sensor modules may vary depending on the sensor type, complexity, and application. In an embodiment, sensors may have additional features or components tailored to their unique sensing principles, application, and other requirements.

According to an embodiment it is an air quality measurement device comprising: a sensor module comprising an array of sensors to sense one or more air quality parameters and generate air quality data; and a communication module to transmit the air quality data to a server; and wherein the array of sensors comprise one or more of a particulate matter sensor, a gas sensor, a nitrogen dioxide sensor, a carbon monoxide sensor, an ozone sensor, a volatile organic compound sensor, a biological sensor, a biochemical sensor; wherein the air quality data comprises carbon dioxide data, volatile organic compounds data, particulate matter data, humidity data, temperature data, a location data, and a time stamp; wherein the air quality measurement device is configured to interface with a user device; and wherein the air quality measurement device is configured for measuring air quality at a geographic location.

According to an embodiment of the system, the sensor module comprises a sensor array with a plurality of air quality sensors, comprising a gas sensor, a particle sensor, and an environmental sensor. According to an embodiment of the system, the one or more air quality parameters comprise CO₂, volatile organic compounds, particulate matter, pathogens, temperature, humidity, and airflow.

According to an embodiment of the system, the sensor module comprises a global positioning system (GPS) sensor configured to generate the geographic location comprising longitude and latitude data. According to an embodiment of the system, the sensor module is configured to monitor the air quality of the geographic location. According to an embodiment of the system, the sensor module further comprises a power management module comprising a sleep mode to conserve battery life when the sensor module is not in use.

According to an embodiment of the system, the sensor module comprises a protective casing comprising shock-absorbing materials to protect the sensor module from accidental drops and impacts. According to an embodiment of the system, the sensor module is portable. According to an embodiment of the system, the sensor module comprises a location determination module comprising GPS.

According to an embodiment, wherein the air quality data is sent to the cloud based application by a smartphone which collects the air quality data from the sensor module using Bluetooth® connection, wherein a location data from the smartphone is also sent to the cloud based application.

According to an embodiment, it is an air quality measurement device comprising: a sensor module for detecting and measuring one or more air pollutants and generating air quality data; a location sensor for identifying a geographic location; a data processing unit for analyzing the air quality data and generating an air quality rating; a display module for displaying air quality rating to a user; a power management module for managing power usage in the sensor module; and a housing configured to house the air quality measurement device and minimize shock. According to an embodiment of the air quality measurement device, the air quality measurement device is portable. According to an embodiment of the air quality measurement device, the air quality measurement device comprises a compact housing for easy portability and shock absorption. According to an embodiment of the air quality measurement device, the air quality measurement device comprises a protective case for safe storage and transportation.

According to an embodiment of the air quality measurement device, the air quality measurement device comprises a rechargeable battery configured for extended use. According to an embodiment of the air quality measurement device, the air quality measurement device comprises a communication module for transmitting the air quality data to a remote device.

Cloud Based Application 104: In an embodiment, Cloud-based application 104 comprises several components that work together to deliver the desired functionality of air quality monitoring. In an embodiment, Cloud-based or Cloud-native application is developed in AWS® comprising several components that work together to provide scalable, reliable, and flexible services.

Compute Resources: Cloud-based applications leverage compute resources to process and execute application logic. Resources comprises virtual servers, containers, serverless functions, or specialized computing services for specific tasks. In an embodiment, AWS compute services, including Amazon Elastic Compute Cloud (EC2) for virtual servers, AWS Lambda for serverless computing, and Amazon Elastic Container Service (ECS) or Amazon Elastic Kubernetes Service (EKS) for containerized applications are configured for the application of air quality monitoring system.

Data Storage: Cloud-based applications require a storage component to store and retrieve data. This can include relational databases, NoSQL databases, file storage systems, or object storage services. In an embodiment, AWS storage services, such as Amazon Simple Storage Service (S3) for scalable object storage, Amazon Elastic Block Store (EBS) for persistent block storage, and Amazon Glacier for long-term archival storage are configured for the Air quality monitoring system. In an embodiment, AWS managed database services, for example, Amazon Relational Database Service (RDS) for relational databases, Amazon DynamoDB for NoSQL databases, and Amazon Redshift for data warehousing are configured for organizing and analyzing the data.

Networking and Connectivity: Cloud-based applications rely on networking components to facilitate communication between different components, as well as with external systems or services. This includes networking infrastructure, load balancers, firewalls, and secure connections. In an embodiment, AWS networking services are configured for building and managing network infrastructure, including Amazon Virtual Private Cloud (VPC) for isolated virtual networks, AWS Direct Connect for dedicated network connections, and Elastic Load Balancing for distributing incoming traffic. Furthermore, AWS Amazon CloudFront, a content delivery network (CDN), is configured to deliver content, for example, air quality information, AQI, AQI ratings, etc. to the users with low latency and high transfer speeds.

User Interface (UI): The user interface component provides the presentation layer through which users interact with the application. It may include web interfaces, mobile applications (mobile apps), or other client-side interfaces.

Application Logic: This component contains the core business logic and algorithms that drive the application's functionality. It processes user requests, performs calculations, and manipulates data.

Application Programming Interfaces (APIs): APIs enable communication and data exchange between different components of the application, as well as with external systems. They define the interfaces and protocols through which different services interact.

Security and Access Control: Security components ensure the application's integrity, confidentiality, and availability. They include authentication mechanisms, encryption, access control, and other security measures to protect data and resources. In an embodiment, AWS Identity and Access Management (IAM), AWS Web Application Firewall (WAF), and AWS Shield for DDoS protection are configured to enable centralized management of user access, permissions, and authentication to AWS resources.

Monitoring and Logging: Cloud-based applications often incorporate monitoring and logging components to track performance, detect issues, and collect operational data. This enables administrators to analyze application behavior, identify bottlenecks, and troubleshoot problems. In an embodiment, AWS services like Amazon CloudWatch are configured for monitoring and collecting metrics, AWS CloudTrail is configured for auditing and tracking API activity, and AWS Systems Manager is configured for managing and automating operational tasks.

Scalability and Elasticity: Cloud-based applications can scale resources up or down based on demand. Components such as auto-scaling groups, load balancers, and resource management tools enable the application to handle varying workloads efficiently.

Integration and Messaging: Integration and messaging components in a cloud-based environment enable connecting and coordinating different systems, services, and components within an application or with external systems. They enable seamless data exchange, facilitate system interoperability, support event-driven architectures, and automate business processes. These components are configured for enabling efficient communication, data synchronization, and workflow coordination, fostering seamless integration and collaboration in the cloud environment. In an embodiment, AWS services such as Amazon Simple Queue Service (SQS) are configured for message queuing, Amazon Simple Notification Service (SNS) are configured for pub/sub messaging, and AWS Step Functions are configured for building serverless workflows.

DevOps and Continuous Integration/Continuous Deployment (Cl/CD) Tools: These components automate application deployment, configuration, and management processes. They facilitate collaboration among development, operations, and deployment teams, enabling faster and more reliable software delivery.

Data Processing Module 106: In an embodiment, data processing module 106 comprises components that handle data input, transformation, processing algorithms, data storage, error handling, and performance monitoring. These components work in tandem to facilitate the efficient processing and transformation of data, ensuring its accuracy and usefulness for generating insights or facilitating further actions. The specific components used within a data processing module depend on the specific requirements and goals of the data processing system at hand. A data processing module comprises a collection of components that work together to process and transform data. It comprises components for data input, which collects data from various sources, and data transformation, which cleans, filters, and normalizes the data. The module also includes artificial intelligence and predictive analytics-based algorithms for performing computations and analyses on the data, along with components for storing intermediate or processed data. Additionally, it encompasses components for handling errors and logging, ensuring smooth processing and identifying issues. Scalability and parallel processing components of the system are enabled for efficient handling of large data volumes, while monitoring and performance optimization components help track performance metrics and enhance processing efficiency.

According to an embodiment of the system, the data processing module comprises an artificial intelligence engine comprising a machine learning algorithm for identifying and classifying the one or more air pollutants. According to an embodiment of the system, the machine learning algorithm is configured to analyze the air quality data collected from the sensor module, air quality monitoring stations and satellite imagery to identify patterns, to predict a pollution level and forecast air quality in the geographic location.

Communication Module 108: In an embodiment, the sensor module is a sensor device enabled for (Internet of Things) IoT-based communication with another IoT device via the communication module 108. In an embodiment, the sensor module is configured for communication with a server via the communication module 108. Communication module 108 comprises various components that work together to ensure reliable and efficient data transfer. In an embodiment, the communication module is configured for supporting standard IoT connectivity protocols like Message Queuing Telemetry Transport (MQTT), Constrained Application Protocol (CoAP), and HTTP(S) or the like to facilitate communication between IoT devices and the server. HTTP(S) refers to the combination of Hypertext Transfer Protocol (HTTP) and Secure Sockets Layer (SSL) or Transport Layer Security (TLS) encryption. It is an extension of the HTTP protocol that adds a layer of security to the communication between clients and servers. Communication module includes features for device management, enabling device registration, authentication, and provisioning, ensuring secure and authorized communication.

Security plays a vital role, and the communication module incorporates encryption protocols like Secure Sockets Layer (SSL)/Transport Layer Security (TLS) to safeguard data during transmission from unauthorized access or tampering. To handle communication in unreliable or intermittent network conditions, a message broker or message queue component is employed for reliable and asynchronous message delivery. Additionally, data compression and optimization mechanisms are also implemented to reduce bandwidth usage and minimize network traffic, particularly in bandwidth constrained IoT environments.

For efficient communication, quality of service (QoS) support is enabled to allow for prioritizing critical or time-sensitive data, and protocol bridging components facilitate seamless communication between devices using different protocols. The module may provide APIs or Software Development Kits (SDKs) for easy integration of IoT devices with the server, simplifying development efforts. Error handling and logging components help detect and handle communication failures, while logging relevant information for troubleshooting purposes. Scalability and load balancing mechanisms are enabled to ensure the communication module may handle a large number of IoT devices efficiently.

Communication module enables communication with a server, encompassing connectivity protocols, device management, security features, message queuing, data optimization, QoS support, protocol bridging, API integration, error handling, logging, and scalability mechanisms. The combination of these components empowers seamless and secure communication between IoT devices and the server, enabling reliable data transfer and facilitating the implementation of IoT solutions.

According to an embodiment of the system, the communication module is enabled for communication using one of a wired connection and a wireless connection. According to an embodiment of the system, the communication module comprises one or more of a Wi-Fi, a Bluetooth, and a cellular connectivity for data reception and data transmission.

Artificial Intelligence (AI) Module 110: In an embodiment, an Artificial intelligence (AI) module 110, comprising machine learning, may be used to enhance the capabilities of the sensor module in data analysis and pattern recognition. AI modules may be used to analyze the data collected by sensor modules to identify patterns, trends, and anomalies. Machine learning algorithms may be trained to recognize specific patterns associated with certain conditions or events, allowing for more accurate and automated detection of environmental changes or target substances. AI modules may further be configured to optimize sensor performance by calibrating sensor readings in real-time and for adaptive performance. Machine learning algorithms may continuously learn from sensor data and adjust calibration parameters, compensating for environmental factors, sensor drift, or aging, thereby improving accuracy and reliability.

In an embodiment, an AI module may be configured for predictive analytics. By analyzing historical sensor data, the AI module may develop predictive models to forecast air quality trends, pollution levels, or potential hazards. This information may be valuable for proactive decision-making, resource allocation, and implementing preventive measures to mitigate air pollution risks. The AI module may be coupled with the control module to implement the preventive measures.

AI modules may further be used in data fusion and integration. The AI module, with the help of cloud networks, may gather and integrate data from multiple sensors, both within a single sensor module and across different sensor modules or networks. By fusing data from various sources (similar sensor modules or from other air pollution monitoring sensors or stations), AI will be able to provide a more comprehensive and holistic view of the air quality conditions. In an embodiment, it may be enabled for an analysis for understanding and assessment of pollution sources based on the sensor readings across multiple locations.

In an embodiment, an AI module may dynamically adjust the sampling frequency or location of sensor measurements based on real-time data analysis and the battery capacity. This adaptive sampling approach optimizes resource utilization and ensures that sensor resources are focused on areas or time frames of higher interest or potential pollution events. For example, if the pollution rate is high, then the sampling frequency may be increased to continue monitoring.

In an embodiment, the sensor module coupled with the AI module may enable the fault detection for sensor maintenance. AI modules may detect sensor malfunctions or anomalies by monitoring sensor data patterns. Machine learning algorithms can identify deviations from expected behavior, flagging potential issues and triggering maintenance or recalibration activities to ensure the sensor modules operate reliably.

In an embodiment, AI modules with machine learning are used to enhance sensor modules by enabling advanced data analysis, adaptive performance optimization, predictive analytics, data fusion, adaptive sampling, and fault detection, ultimately improving the accuracy, efficiency, and reliability of the sensor-based monitoring systems.

User Device 112: A user device 112 refers to any electronic device used by an individual to access or interact with digital services, applications, or content. In an embodiment, it comprises at least one of desktop computers, laptops, and notebooks, which are commonly used for tasks such as web browsing, document processing, multimedia consumption, and software applications. In an embodiment it may comprise mobile devices like smartphones and tablets, which offer mobility and portability, allowing users to access a wide range of applications, communication services, and internet browsing on the go. In another embodiment, it may include wearable devices which include smartwatches, fitness trackers, augmented reality (AR) glasses, and other devices that can be worn on the body. They provide functionalities including but not limited to air pollution or air quality tracking or rating, notifications, location recommendations, and hands-free access to information via a user interface 112-1. In another embodiment, it is an Internet of Things (IoT) device, which are connected devices embedded in everyday objects which enable users to monitor and control various aspects of the environment through network connectivity. In an embodiment, the user device may be an infotainment system of a transport vehicle which the user may be driving or occupying.

According to an embodiment of the system, the user device comprises a display configured to display the first air quality index.

Application Program: An application program, commonly known as an app, is a software program that performs specific tasks or provides specific functionalities on a computing device. It comprises various components that work together to deliver an intended functionality, such as air quality tracking and rating, recommendation of an alternate location and/or a control command for purifying the air in and around the current location. The user interface (UI) component provides visual and interactive elements through which users can interact with the app. The business logic component contains the core algorithms and rules that define the app's behavior. Data management handles tasks related to storing, retrieving, and manipulating data, while the networking components enable communication with remote resources and services. Security components ensure the protection of user data and secure communication. Integration components enable the app to interact with external services and APIs, while the notification component manages the delivery of updates and alerts to users. These components collectively form the foundation of an application program, allowing it to provide the desired functionalities and deliver a seamless user experience.

According to an embodiment of the system, the application program generates an alert and notifies a user via one or more of an audio cue, a visual cue, a tactile cue, and a text message.

Alert Module 114: An alert module 114 of the system or application is configured for generating and delivering notifications or alerts to users. Its function is to promptly inform individuals about one or more air pollutants, air quality data and ratings, air quality changes, or critical environmental issues that require their attention, for example, a bad weather or an endemic alert at the geographic location. The alert module is configured for monitoring various triggers or conditions within the system or application. These triggers can include system pollutant level threshold, system or sensor failures, errors, security breaches, threshold breaches, environmental updates, or any event that requires immediate attention or action of the user.

Once a trigger is detected, the alert module generates an alert or notification which can take various forms depending on the system's design and the intended audience. This can include visual alerts like a color change, audio alerts like a beep, tactile alerts like vibration in user devices, email notifications, Short Message Service (SMS) messages, mobile app push notifications, or even automated phone calls. The alert module may also include features such as pollution severity levels, future predictions for the location. The alert module may further comprise escalation processes, and notification preferences to ensure that alerts are appropriately prioritized, routed to the right individuals or groups, and delivered through the preferred communication channels. By utilizing an alert module, system administrators, operators, or users can stay informed about critical pollutant and environmental events or issues in real-time. This enables them to take immediate action, troubleshoot problems, address security threats, or respond to important updates, and ensure the smooth operation of the system or application.

Control Module 116: In an embodiment, a control module 116 for an air purification system may be coupled and configured for managing and regulating the operation of the system to ensure efficient and effective air cleaning. The module comprises several components that work together to control the purification process.

In an embodiment, the control module interfaces with sensors to monitor air quality parameters such as particulate matter (PM), volatile organic compounds (VOCs), humidity, temperature, and gas concentrations. These sensors provide real-time data on air pollution levels and enable the system to adjust its operation accordingly. In another embodiment, the controller is the central processing unit of the control module. It receives input from the sensors, analyzes the data, and determines the appropriate actions to be taken. The controller applies predefined algorithms and logic to make decisions based on the air quality readings. The controller sends a signal to the actuators. The actuators are devices that receive signals from the controller and initiate specific actions. In the context of an air purification system, actuators control the operation of components such as fans, filters, valves, and motors. The controller adjusts the speed of the fan, regulates airflow, activates or deactivates filters, and controls the overall system operation. The control module may interface with the user interface of the user device, such as a display panel or a mobile application, to allow users to interact with the system. The user interface provides information about the air quality status and ranking, system settings, and allows users to adjust preferences, set timers, or activate special modes. In an embodiment, the user may send the control signal to the indoor space central authority. Based on the received signal, the central authority may enable the signal to reach the control unit of the air filtration system for the overall indoor space.

The control module may further interface with the communication module that enables connectivity with external devices or networks. This allows for remote monitoring and control of the air purification system, integration with smart systems, or communication with central monitoring stations. The working of the control module involves continuously monitoring the air quality through sensors, analyzing the data using the controller of the control module, and activating the appropriate actuators to adjust the purification system's operation. For example, if the air quality deteriorates, the controller may increase the fan speed, activate additional filters, or adjust airflow to enhance the air cleaning process. The user interface provides feedback and control options for users to customize settings and monitor the system's performance.

In another embodiment, the user may have a personal air purification system, which can influence the purification of the air in his vicinity. In an embodiment, the control module is configured to maintain optimal air quality by dynamically adjusting the air purification system's parameters based on real-time sensor data and user preferences.

Location and Time Module 118: A location and time module 118 of the system, or application, deals with determining and managing location-related information and time-related data. The location aspect of the module involves accessing and processing data related to the geographical position of a user device, air quality measurement device, or user. This can be done through various means, such as GPS (Global Positioning System), Wi-Fi signals, cellular networks, or Internet Protocol (IP) address geolocations. The module may provide functions to retrieve current location coordinates, track movement, calculate distances, or display maps.

The time aspect of the module focuses on managing and manipulating time-related data. It includes functions to obtain the current date and time, handle time zone conversions, perform date and time calculations, and synchronize with global time references, such as atomic clocks or network time protocols.

Combined, the location and time module enables applications to incorporate location-aware features, such as location-based services, geofencing, mapping, navigation, or weather information. It also facilitates time-dependent functionalities, such as scheduling, event management, time-based notifications, or time-sensitive data analysis. This module is enabled for context-awareness, real-time updates, or location-dependent functionalities. It allows the system to integrate location and time data into the application program and data, enhancing user experiences, enabling personalized services, and facilitating accurate data processing based on geographical and temporal factors.

There are air quality rating sensors that are available to detect the air quality of an area. There are also products such as air purifiers which have an integrated air sensor module to detect the air quality. However, there is no publicly available database for people to view the air quality information of indoor spaces they would like to visit, live, or work at. Further there is no integration of air quality prediction and appropriate mitigation actions. The air quality of most indoor spaces is not measured and rated. It is largely left to the individual perception of the inhabitants of the space. In an embodiment, the system quantitatively rates and makes available the air quality of indoor locations to the inhabitants of the space and people considering inhabiting the space to access the air quality information. This may solve the problem of poor indoor air quality and allow people to want to visit by encouraging the inhabitants to improve the indoor air quality of the space (through improving ventilation and implementing air purifiers).

According it an embodiment, it is a system comprising, a sensor module configured to detect and measure one or more air pollutants at a geographic location via one or more sensors and generate air quality data; a cloud based application comprising a data processing module configured to receive, via a communication module, the air quality data and derive a first air quality index; a user device comprising an application program configured to receive, via the communication module, the first air quality index; the application program configured to suggest an alternate location having a second air quality index, wherein the alternate location has better air quality than the geographic location; and wherein the air quality data comprises one or more air quality parameters, the geographic location corresponding to the one or more air quality parameters and a time stamp; and wherein the system is configured to provide real-time data of an air quality of the geographic location and the alternate location.

A second value of the second air quality index is lower than a first value of the first air quality index is indicative that the air quality at the alternate location is better than the geographic location.

According to an embodiment of the system, the system is configured to generate the first air quality index by crowdsourcing via cloud computing. According to an embodiment of the system, the system is configured to generate the second air quality index by crowdsourcing via cloud computing. According to an embodiment of the system, the system is configured to generate an air quality index for a future period using a prediction model based on current air quality information and a history of air quality information.

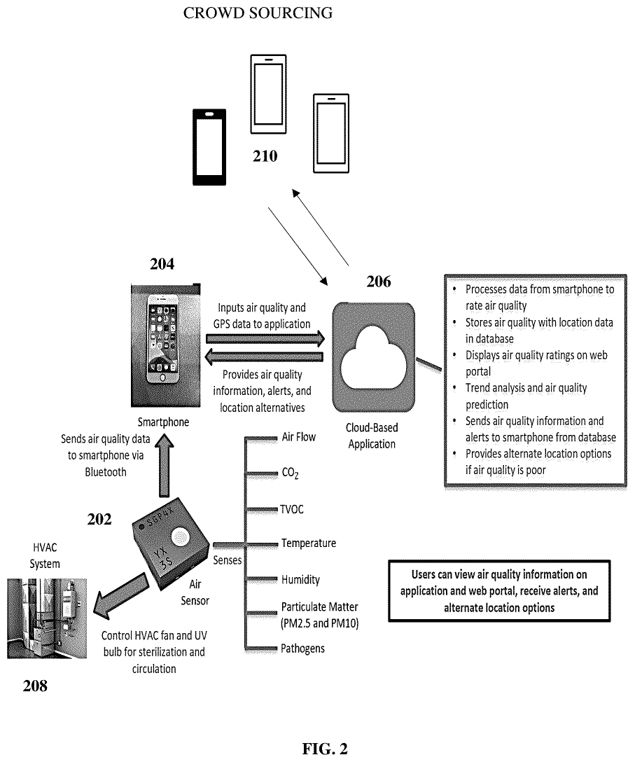

FIG. 2 shows various components of the system configured for measuring air quality using crowdsourcing according to an embodiment. In an embodiment, the system is configured for sensing indoor air quality, however the system may also be configured for air quality and pollution monitoring of the environment for outdoor air. The invention comprises technology modules: (1) sensor module 202, (2) smartphone application 204, (3) cloud infrastructure 206. The sensor module 202 is an electronic device comprising a microcontroller that operates and manages a set of sensors. The sensor module 202 includes sensors that sense temperature, humidity, particulate matter, VOCS (volatile organic compounds), carbon dioxide, air flow, and pathogens. The smartphone application is the interface to the air sensing module to capture the measurements detected by the individual sensors in the sensor module 202. The smartphone application will also interpret the sensor data and display it in the smartphone application as discrete values for each parameter and will also rate the air as good, moderate, or bad based on the government approved AQI ratings which are determined based on the parameters detected by the sensor module 202 (humidity, particulate matter, VOCS, carbon dioxide, air flow, and pathogens). The smartphone application 204 also captures the location data (of the location the air quality data is gathered) using the Google Maps API as GPS coordinates of the location. Once the air quality and GPS data are gathered, they are transmitted to the cloud infrastructure 206 by the smartphone application via cellular connectivity. The cloud infrastructure 206 comprises a cloud-based software application and database configured on AWS (Amazon Web Services). The cloud infrastructure 206 receives air quality data and GPS data transmitted to it by the smartphone application. The cloud infrastructure software matches the GPS coordinates with the address and location name using Google Location Services. Once this has been done, the cloud infrastructure software then matches the location data with the air quality data and stores it in the AWS database. Once this has been done, the air quality of the location becomes available to view and perform further analysis such as trend analysis, prediction, alert warning, and corrective action.

In an embodiment, the system may be used to execute the following steps of a method for sensing the air quality:
i. User carries an air sensing device and smartphone at a location where he is visiting (Ex., a restaurant, a business location, etc.)
ii. Air sensing device senses the air quality of the location.
iii. Air sensing device transmits air quality data to the smartphone.
iv. Smartphones gather GPS location data and match it with air quality data (on a smartphone app). In an embodiment, the sensor may itself comprise the GPS module to gather the location data.
v. Smartphones transmit location data and air quality data onto cloud-based applications.
vi. Air quality data of location is available to users on the smartphone application and on the web to view.
vii. Devices that are subscribed to the service will receive air rating information and alerts based on real-time air quality at a location any time they are at the location where data has previously been collected.

According to an embodiment of the method, the method further comprises controlling an air purification system via a control module based on the air quality rating to improve the air quality. According to an embodiment of the method, the air quality rating is transmitted to a second user device for real-time access of the air quality of the geographic location by a second user.

According to an embodiment of the method, the method further comprises a recommendation unit configured to recommend location along with the air quality to make informed decisions about an outdoor activity and a travel route based on the air quality rating. According to an embodiment of the method, the air quality data is analyzed over time to identify patterns and trends, enabling proactive measures to be taken to address potential air quality issues.

According to an embodiment of the method, the method is configured for predicting the air quality rating in a region comprising: collecting historical air quality data from one or more sources of the sensor module; using a machine learning algorithm to identify a pattern and a trend in the historical air quality data; generating a predictive model for air quality for a future period; updating the predictive model with real-time air quality data; disseminating the air quality for the future period to the public; and issuing alerts and recommendations based on a predicted air quality.

According to an embodiment of the method, the method further comprises a strategy to improve air quality comprising one or more of controlling a ventilation system, an air purifier, and implementing a source control measure to reduce emissions. According to an embodiment of the method, the server comprises a web-based dashboard for visualizing and analyzing the air quality data from multiple geographic locations.

According to an embodiment of the system executing the method, the sensor module comprises a rechargeable battery and includes a fast-charging feature for rapid recharging of the sensor module. According to an embodiment of the method, the user device comprises a display module comprising a touch screen interface for a user interaction. According to an embodiment of the method, the sensor module comprises a protective case comprising a built-in charger for recharging the sensor device while it is stored in the protective case.

According to an embodiment of the method, the method is configured for monitoring air quality in a building comprising: installing one or more sensor modules in an indoor space; collecting the air quality data from the one or more sensor modules; transmitting the air quality data to a central server; analyzing the air quality data to identify potential source of an air pollution; implementing a strategy to improve air quality; and re-evaluating air quality over time to determine an effectiveness of the strategy.

According to an embodiment, it is a method for measuring air quality comprising, detecting, and measuring one or more air pollutants using a sensor array; analyzing sensor data to determine an air quality index; presenting the air quality index to a user through a user interface; transmitting the air quality index to a remote device; and using the air quality index to decide on a control comprising a ventilation control and an air purification control of an indoor environment.

The air sensing module and the smartphone application developed by Air Rating, Inc. are provided to users of the service. The users will comprise any individual of the public interested in the air quality of the indoor space they live, work, or plan to visit. The users who choose to participate are the source of the air quality information (crowdsourcing). As participating users carry their air quality sensor and smartphone to a location, the air quality and location data is transmitted to the cloud (via the smartphone application) allowing for all users to view the air quality of the location online (on the smartphone application and web portal). The users who may use the air quality services are:
viii. Business owners who want to advertise air quality of their business to attract customers;
ix. Customers (of local businesses) who assess air quality of businesses to determine if indoor air quality is safe for them to visit;
x. Employees who assess air quality of workplace to determine if indoor air quality is safe;
xi. Employers who advertise air quality to attract and assure employees;
xii. Property owners who want to advertise air quality of property to attract tenants;
xiii. Tenants who want to assess air quality of their living space to confirm it is safe;
Many such needs will arise as the services are offered towards determining and quantifying air quality.

The indoor air quality rating system will be used as follows:
xiv. User has air sensing device and smartphone at location (restaurant, business, etc.);
xv. Air sensing device senses air quality of location;
xvi. Air sensing device transmits air quality data to smartphone;
xvii. Smartphone gathers location data via GPS data;
xviii. Smartphone input's location data and air quality data onto cloud-based application;
xix. Air quality data of location is available to all users of the service on the web portal and smartphone application;
xx. Periodic updates of air quality information and alerts will be given to subscribed users of their respective locations;

An application residing in the smartphone 204 (smartphone application or App) is the interface to the air sensing module to capture the measurements detected by the individual sensors in the sensor module 202. The application residing in the smartphone 204 will also interpret the sensor data and display it in the application residing in the smartphone 204 as discrete values for each parameter and will also rate the air as good, moderate, or bad based on the government approved AQI ratings which are determined based on the parameters detected by the sensor module 202 (humidity, particulate matter, VOCS, carbon dioxide, air flow, and pathogens). The application residing in the smartphone 204 also captures the location data (of the location the air quality data is gathered) using the GPS coordinates of the location. The GPS data of the location is gathered by the application residing in the smartphone 204 using cellular connectivity. Once the air quality and GPS data are gathered, they are transmitted to the cloud infrastructure 206 by the application residing in the smartphone 204 via cellular connectivity.

The cloud infrastructure 206 comprises a cloud-based software application 204 and database configured on AWS (Amazon Web Services). The cloud infrastructure 206 receives air quality data and GPS data transmitted to it by the application residing in the smartphone 204. The cloud infrastructure 206 software matches the GPS coordinates with the address and location name using Google Location Services. Once this has been done, the cloud infrastructure 206 software then matches the location data with the air quality data and stores it in the AWS database. Once this has been done, the air quality of the location becomes available to view and perform further analysis such as trend analysis, prediction, alert warning, and corrective action (alternate location suggestions). The location data, air quality of the location, trend analysis, and predictions are stored in the AWS database. This database is accessible through a web portal login created by users at the time of subscribing to the service. This same database is accessed by the application residing in the smartphone 204 when the user chooses to review the air quality of a location on their application residing in the smartphone 204 graphical user interface. The application residing in the smartphone 204 may also display alert notifications in the event of air quality rated as bad being recorded in the database for the GPS location of the smartphone.

The sensor module is an electronic hardware with custom software. The hardware comprises a microcontroller that operates and manages a set of sensors. The sensor module will include sensors that sense temperature, humidity, particulate matter, VOCS (volatile organic compounds), carbon dioxide, air flow, and pathogens. The microcontroller used is the ESP-32S for controlling the sensor modules and transmitting the air quality data to the smartphone via Bluetooth. The microcontroller also controls a relay which turns the fan of Heating, Ventilation and Air Conditioning (HVAC) system 208 on or off based on the need to increase circulation (as sensed by air flow sensor). The humidity and temperature sensor used is the DHT11 sensor. The carbon dioxide and VOC (volatile organic compound) sensor is Sensiron's SGP30® sensor. The PM1.0, PM2.5, and PM10 sensor is the Grove HM3301® sensor. The air flow is sensed using Omron's DGF-A5 MEMS air flow sensor.

In an embodiment, detection of airborne pathogens in air is accomplished without requiring sample collection in liquid or solid matrices. According to one aspect, a method for airborne detection of target analytes, such as biological or chemical substances, is accomplished by exposing a sensing apparatus to the airborne analyte. A recognition entity, located on the sensing apparatus, binds to the analyte and is detectable while the analyte is present in a gas. Specific example recognition entities include chemical coatings for the detection of chemicals and immobilized antibodies for the detection of biologicals. In one aspect of the invention, a sensing apparatus includes a sensor that includes a piezoelectric layer, a non-piezoelectric layer, and a recognition entity located on either of the two layers. In a first embodiment, the sensor is a cantilever assembly that is fixed at only one end. Here, the piezoelectric layer is connected to a base and the non-piezoelectric layer is attached to the end of the piezoelectric layer in an overlap fashion. Electrodes are attached to the piezoelectric layer and are electrically driven to excite the piezoelectric layer to resonance. A recognition region on the non-piezoelectric layer attracts the analyte when exposed in an airflow and causes a change in mass of the cantilever that is formed by the combination of the piezoelectric and non-piezoelectric layers and the recognition area. The change in resonant frequency when the analyte is attached compared to a baseline resonant frequency is determined and the frequency shift is indicative of the amount of analyte held on the recognition entity. Methods and systems related to detecting airborne species as discussed in U.S. Pat. No. 8,171,795B1 titled, “Self-exciting, self-sensing piezoelectric cantilever sensor for detection of airborne analytes directly in air” are incorporated herein in their entirety. Similarly, methods and systems related to detecting airborne particles, airborne biological particles, and systems of monitoring air quality as discussed in U.S. Pat. No. 7,578,973B2, titled, “Devices for continuous sampling of airborne particles using a regenerative surface” are incorporated herein in their entirety.

The cloud infrastructure comprises a cloud-based software application 206 and database configured on AWS (Amazon Web Services). The cloud infrastructure receives air quality data and GPS data transmitted to it by the application residing in the smartphone 204. The cloud infrastructure software matches the GPS coordinates with the address and location name using Google Location Services. Once this has been done, the cloud infrastructure software then matches the location data with the air quality data and stores it in the AWS database. Once this has been done, the air quality of the location becomes available to view and perform further analysis such as trend analysis, prediction, alert warning, and corrective action. The data stored in the AWS database is analyzed to provide guidance on future air quality via rating predictions based on past air quality trends in correlation with the most recent air quality data of the location.

In an embodiment, the users of the air quality rating system may be provided a portable sensor with a form factor of 4×3×1.5 inches. The sensor module 202 which is portable is carried along with the user's smartphone. In another embodiment, the sensor system may be part of the smartphone.

Users subscribing to the air quality alert services are provided notifications when they are at a location with a poor air quality rating. The user's location is determined using the Google Maps® API. This location data is looked up in the air quality database. If the air quality is below the desirable level, an alert is provided as a notification on the user's smartphone. The user has the option to enable this by allowing push notifications for this application on the smartphone 204. When the air quality of the location of the user is poor, a notification is sent to the user's smartphone with vibrations, light, and a sound warning on the phone (when enabled by the user on smartphone). Also, when the user subscribes to the service, he is required to enter his phone number and may opt to enter his email address. Text messages and emails are also sent to the user if he/she is in a location with poor air quality (emails and text messages must be enabled by the user on the smartphone app).

The air quality data of a specific location is logged into the AWS database. The air quality parameters, specifically particulate matter and air flow are analyzed to estimate the probable status of the air quality for the next hour enabling users to base their decisions to remain at the location or to visit the location. The rate of change of the particulate matter and air flow are the basis of predicting the quality of air over the next hour. Air flow and particulate matter readings are captured in intervals of 5 minutes to determine the rate of change of the particulate matter and air flow. The rate of change of the particulate matter in correlation with the air flow is used to predict the quality of air over the next hour.

In an embodiment, a corrective action is configured to provide alternate location recommendations based on GPS data and alternate locations. In the event of the air quality rating being low in a certain location, a user will be provided locations of the same kind of business which will include the same category, same franchise, or the next closest alternative. This would be accomplished through the Google Maps and Google Directory APIs utilized by the cloud infrastructure of the system.

In an embodiment, a corrective action may be suggested by interfacing with HVAC system 208 and filtration system of a building to remedy air quality for indoor areas. The air sensing module of the system is an electronic hardware with custom software. The hardware comprises a microcontroller that operates and manages a set of sensors. The ESP32 microcontroller controls a relay which turns the fan of the HVAC system controller on or off based on the need to increase circulation (as sensed by air flow sensor). The air flow is sensed using Omron's D6F-A5 Micro-electromechanical systems (MEMS) air flow sensor.

In an embodiment, the system provides the air quality of indoor locations. Based on the indoor location air quality, the user can make decisions on where to live, to visit or to work. This process of determining the air quality at an indoor space is via crowdsourcing 210 using multiple portable devices or smartphones 204. Crowdsourcing refers to the practice of obtaining information, services, or solutions by soliciting contributions from a large group of people, typically through an open call or online platform. It involves harnessing the collective data of a diverse crowd to accomplish air quality determination at a location. Crowdsourcing will make these air quality ratings of these indoor link locations more extensive. People all around the world will be able to review the indoor air quality ratings. The availability of these indoor air quality ratings will create increased awareness of the state of indoor air quality, thus deriving the desire and need, everywhere, to improve indoor air quality.

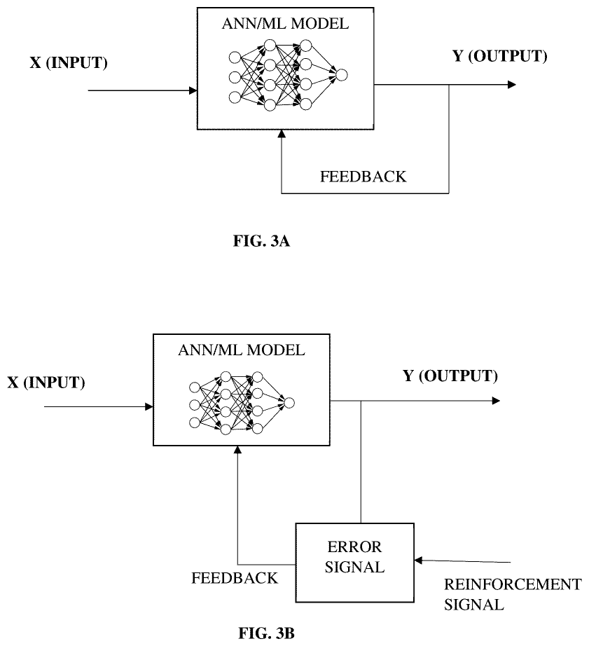

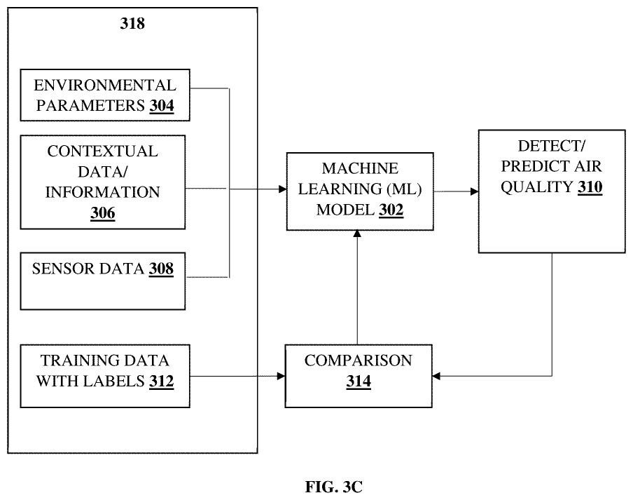

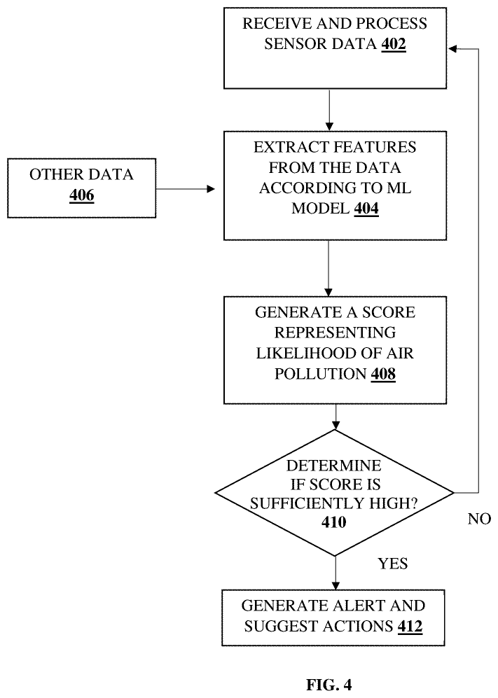

FIG. 3A shows a structure of the neural network/machine learning model with a feedback loop according to an embodiment of the system. Artificial neural networks (ANNs) model comprises an input layer, one or more hidden layers, and an output layer. Each node, or artificial neuron, connects to another and has an associated weight and threshold. If the output of any individual node is above the specified threshold value, that node is activated, sending data to the next layer of the network. Otherwise, no data is passed to the next layer of the network. A machine learning model or an ANN model may be trained on a set of data to take a request in the form of input data, make a prediction on that input data, and then provide a response. The model may learn from the data. Learning can be supervised learning and/or unsupervised learning and may be based on different scenarios and with different datasets. Supervised learning comprises logic using at least one of a decision tree, logistic regression, and support vector machines. Unsupervised learning comprises logic using at least one of a k-means clustering, a hierarchical clustering, a hidden Markov model, and an apriori algorithm. The output layer may predict or detect a health issue and the severity of the health issue based on the input data.

In an embodiment, ANNs may be a Deep-Neural Network (DNN), which is a multilayer tandem neural network comprising Artificial Neural Networks (ANN), Convolution Neural Networks (CNN) and Recurrent Neural Networks (RNN) that can recognize features from inputs, do an expert review, and perform actions that require predictions, creative thinking, and analytics. In an embodiment, ANNs may be Recurrent Neural Network (RNN), which is a type of Artificial Neural Networks (ANN), which uses sequential data or time series data. Deep learning algorithms are commonly used for ordinal or temporal problems, such as language translation, Natural Language Processing (NLP), speech recognition, and image recognition, etc. Like feedforward and convolutional neural networks (CNNs), recurrent neural networks utilize training data to learn. They are distinguished by their “memory” as they take information from prior input via a feedback loop to influence the current input and output. An output from the output layer in a neural network model is fed back to the model through the feedback. The variations of weights in the hidden layer(s) will be adjusted to fit the expected outputs better while training the model. This will allow the model to provide results with far fewer mistakes.

The neural network is featured with the feedback loop to adjust the system output dynamically as it learns from the new data. In machine learning, backpropagation and feedback loops are used to train an AI model and continuously improve it upon usage. As the incoming data that the model receives increases, there are more opportunities for the model to learn from the data. The feedback loops, or backpropagation algorithms, identify inconsistencies and feed the corrected information back into the model as an input.

Even though the AI/ML model is trained well, with large sets of labeled data and concepts, after a while, the models' performance may decline while adding new unlabeled input due to many reasons which include, but not limited to, concept drift, recall precision degradation due to drifting away from true positives and data drift over time. A feedback loop to the model keeps the AI results accurate and ensures that the model maintains its performance and improvement, even when new unlabeled data is assimilated. A feedback loop refers to the process by which an AI model's predicted output is reused to train new versions of the model.

Initially, when the AI/ML model is trained, a few labeled samples comprising both positive and negative examples of the concepts (e.g., bad or good air quality) are used that are meant for the model to learn. Afterward, the model is tested using unlabeled data. By using, for example, deep learning and neural networks, the model can then make predictions on whether the desired concept/s (e.g., air quality that needs to be detected) are in unlabeled images. Each image is given a probability score where higher scores represent a higher level of confidence in the models' predictions. Where a model gives an image a high probability score, it is auto labeled with the predicted concept. However, in the cases where the model returns a low probability score, this input may be sent to a controller (may be a human moderator) which verifies and, as necessary, corrects the result. The human moderator may be used only in exceptional cases. The feedback loop feeds labeled data, auto-labeled or controller-verified, back to the model dynamically and is used as training data so that the system can improve its predictions in real-time and dynamically.

FIG. 3B shows a structure of the neural network/machine learning model with reinforcement learning according to an embodiment of the system.

The network receives feedback from authorized networked environments. Though the system is similar to supervised learning, the feedback obtained in this case is evaluative, not instructive, which means there is no teacher as in supervised learning. After receiving the feedback, the network performs adjustments of the weights to get better predictions in the future. Machine learning techniques, like deep learning, allow models to take labeled training data and learn to recognize those concepts in subsequent data and images. The model may be fed with new data for testing, hence by feeding the model with data it has already predicted over, the training gets reinforced. If the machine learning model has a feedback loop, the learning is further reinforced with a reward for each true positive of the output of the system. Feedback loops ensure that AI results do not stagnate. By incorporating a feedback loop, the model output keeps improving dynamically and over usage/time.

FIG. 3C shows an example block diagram for predicting air quality using a machine learning model according to an embodiment of the system. The machine learning model 302 may take as input any data associated with the geographic location and learn to identify features within the data that are predictive of air quality. The training data sample may include, for example, the one or more air pollutants such as particulate matter (PM), nitrogen dioxide (NO2), sulfur dioxide (SO2), carbon monoxide (CO), and ozone (O3); pathogens that can be present in the air include bacteria, viruses (such as influenza and coronaviruses), fungi, and allergens; environmental parameters 304 including temperature, humidity, air pressure, wind speed and direction, precipitation, solar radiation, noise level, air quality. In an embodiment, it relates to systems and methods that alert a user for a bad air quality or bad environment event or the like and also further capture the health status of the user. The captured data and information are transmitted to the cloud, where the air quality and environmental parameters data is coupled with available public information. Subsequently, the pollutant or AQI index information is transmitted to the user. The systems and methods of the present disclosure may also provide data analytics information that may be used for decision making for the user based on the air quality information. The sensors of the system detect and measure one or more air pollutants and pathogens and transmit the data to the cloud. This real time air quality data, location data, and map that is associated with the geographic area are stored in the cloud. Thus, at all times, the cloud system monitors the data from various user devices, i.e., crowd sourced data.

In an embodiment, the training data sample may also include current contextual information 306 relating to the geographic location, the user who is measuring the air quality, and the purpose for which the user is measuring the air quality. This may include, for example, location of the user (i.e., the sensor), current weather condition, temperature, time of day, traffic conditions in the region, environmental conditions, etc. The system may also garner contextual information 306 from other IoT or nearby devices such as weather monitoring stations. For example, through an application installed in the user device, for example, google maps, location services, calendars, the system may know the details of the passengers, for example, if they are camping, at a birthday party, a wedding ceremony, etc. For example, the users' health conditions may be obtained from an image of the camera where eye pattern is monitored, a sensor where heart rate is monitored, etc. The system may correlate the user health data and air quality from the contextual information. In an embodiment, the application may access other types of application usage data from other applications (including the operating system) installed on the users' device and use them as contextual information 306. In an embodiment, a microphone, installed in the sensor device, mobile device, or in an external device located with the user, may record the noises around the geographic area in the vicinity of the user.

Real-time sensor data 308 may include, for example, video, image, audio, infrared, temperature, sensor data of the one or more air pollutants, etc. that are sensed and read by the sensor module of the system. In an embodiment, the real-time sensor data may be processed using one or more machine learning models 302 trained and based on similar types of data to detect real-time air pollution levels and air quality data. In an embodiment, the real time data from crowdsourcing for the same location may be used to improve the prediction of air quality index. In an embodiment, the data from crowdsourcing may be used to detect a pattern in the Air Quality Index and pollution levels and identify a source of the pollutant. In an embodiment, the historical data at the geographical region combined with real time readings may be used to predict a future air quality level. In an embodiment, other factors, such as environmental initiatives, future developmental project initiatives, may be factored in while predicting the future air quality.

Any of the aforementioned types of data (e.g., environmental parameters 304, contextual information 306, sensor data 308) may correlate with the geographical region, and such correlation may be automatically learned by the machine learning model 302. In an embodiment, during training, the machine learning model 302 may process the training data sample (e.g., environmental parameters 304, contextual information 306, sensor data 308) and, based on the current parameters of the machine learning model 302, detect or predict an air pollution level and air quality 310. The detection or prediction of air quality 310 may depend on the training data with labels 312 associated with the training data sample 318. Predicting air quality refers to predicting a future event based on past and present data and most commonly by analysis of trends or data patterns. Prediction or predictive analysis employs probability based on the data analyses and processing. Detection of air quality refers to air quality that is detected via sensors. Predicted events may or may not turn into reality based on how the turn of events happens. For example, the system based on a developmental initiative, such as construction activities, at the place predict high pollution levels, but upon halting or shifting the constructional initiatives to some other region, may improve the air quality at the place and deviate from the predicted levels.

In an embodiment, during training, a detected or predicted air quality at 310 and the training data with labels 312 may be compared at 314. For example, the comparison 314 may be based on a loss function that measures a difference between the detected or predicted air quality 310 and the training data with labels 312. Based on the comparison at 314 or the corresponding output of the loss function, a training algorithm may update the parameters of the machine learning model 302, with the objective of minimizing the differences or loss between subsequent detections or predictions of the air quality 310 and the corresponding training data with labels 312. By iteratively training in this manner, the machine learning model 302 may “learn” from the different training data samples and become better at detecting air quality 310 due to various pollutants that are like the ones represented by the training data with labels at 312. In an embodiment, the machine learning model 302 is trained using data which is a specific sensor type for which the model is used for detecting air pollution level. In an embodiment, the machine learning model 302 is trained using data which is general to the one or more air pollutants detection and is used for the sensor or device for detecting one or more air pollutants.

In an embodiment, sensor data 308 is associated with a time segment (e.g., 5, 10, 30, or 60 seconds) that may be labeled, which is training data with labels 312 or associated with one or more predetermined event types to represent what transpired or occurred during that time segment. For example, a particular training data sample 318 capturing one or more air pollutants occurring in a geographical region may be labeled to generate training data with labels 312 or associated with a “high pollution” category. For example, a particular training data sample 318 may pertain to a particular incident that occurred in the past.

Using the training data, a machine learning model 302 may be trained so that it recognizes features of input data that signify or correlate to certain event types. For example, a trained machine learning model 302 may recognize data features that signify the likelihood of a pollutant and air quality as an actionable event. In an embodiment, the features may have meaningful interpretations, such as smoke, fog, pollens, allergens, health conditions, etc. In an embodiment, particular pollutants are used as training data or input data for training. Through training, the machine learning model 302 may learn to identify predictive and non-predictive features and apply the appropriate weights to the features to optimize the machine learning model's 302 predictive accuracy. In embodiments where supervised learning is used and each training data sample 318 has one or more air pollutant label to generate training data with labels 312, the training algorithm may iteratively process each training data sample 318 (including environmental parameters 304, contextual information 306, and/or sensor data 308), and generate a prediction of air quality 310 based on the model's current parameters. Based on the comparison 314 results, the training algorithm may adjust the model's parameters/configurations (e.g., weights) accordingly to minimize the differences between the generated prediction of air quality 310 and the corresponding training data with labels 312. Any suitable machine learning model and training algorithm may be used, including, e.g., neural networks, decision trees, clustering algorithms, and any other suitable machine learning techniques. Once trained, the machine learning model 302 may take input data, associated with a geographic location and air pollutant, and output one or more predictions that indicate a likelihood that a bad air quality event has occurred.

FIG. 4 shows an example flow chart for predicting air quality using a machine learning model according to an embodiment of the system. The system may receive data associated with sensor output(s) from one or more sensors in the sensor module as shown at 402. Any suitable type of sensor may be used to gather data pertaining to the air quality. A sensor output may be, for example, images, videos, audios, infrared measures, temperature measures, global positioning system (GPS) data, particulate matter (PM), nitrogen dioxide (NO2), sulfur dioxide (SO2), carbon monoxide (CO), ozone (O3), bacteria, viruses (such as influenza and coronaviruses), fungi, allergens or any other information measured or detected by sensors. In an embodiment, a sensor output may be the result of one or more sensors capturing environmental information associated with the geographic location. In an embodiment, data associated with sensor output may include the raw sensor output (e.g., the images, videos, audios, etc.) and/or data derived from processing the sensor output. For example, users' computer system, a user device, or a cloud/network system may process the sensor data and generate derivative data. For example, derivative data from audio may include data representing levels of the pollution. As another example, derivative data from image, video, or data may include data representing pollutant levels, etc. The system may receive any data associated with the sensor output from sensors, including raw output and/or any derivative data. In an embodiment, the system may process the received data and identify any actionable event of interest, i.e., generating an alert signal, a control signal for pollution decrease, and/or suggest an alternate location using a machine learning model trained using a set of training data.

As shown at step 404, the system may extract features from the received data according to a machine learning model that is able to automatically do so based on what it learned during the training process. In an embodiment, appropriate weights that were learned during the training process may be applied to the features. At step 408, the machine learning model, based on the features of the received data, may generate a score representing a likelihood or confidence that the received data is associated with a particular alert level or pollution level, e.g., high air pollution alert due to endemic and toxins present in the air, etc. As shown at step 410, the system may determine whether the score is sufficiently high relative to a threshold or criteria to warrant certain action. If the score is not sufficiently high, thus indicating that the detected event may not have actually occurred (in other words, a false-positive), the system may return to step 402 and continue to monitor subsequent incoming data. On the other hand, if the score is sufficiently high, then at step 412 the system may generate an appropriate alert and/or determine an appropriate action/response, i.e., suggesting an alternate location. In an embodiment, the system may send alerts to appropriate recipients based on the detected event types.

For instance, an alert is generated, and a message is sent to nearby connected devices via the mobile application. In an embodiment, an alert comprising air pollution data and health emergency due to pollution may be automatically sent to a nearby pollution control board. In an embodiment, along with the alert, the system may advise or suggest an alternate location.

In an embodiment, the system may repeat one or more steps of the method of FIG. 4, where appropriate. In an embodiment, the steps 402 to 412 may be performed by the system, any combination of those steps may be performed by any other computing system, for example, a remote network or a cloud network. In an embodiment, where machine learning models are used for making such determination, the system may transmit a trained machine learning model to the computing system of the user device or air sensing device to allow event-detection to be made locally. This may be desirable since sensor data may be overly large to transmit to the remote system in a timely fashion. If the machine learning model takes as input other data 406 (e.g., the historic pollution/air quality data, etc.) such information may be made available to the computing device executing the machine learning model. In an embodiment, the local computing device may send the event-detection determination to the system and let it perform the appropriate actions (e.g., generate alerts, etc., as described with reference to step 412).

In an embodiment, the system is provided, the AI module may gather data from various sources such as air quality sensors, weather stations, satellite imagery, and environmental databases. This data collection process can be automated and continuous, ensuring a comprehensive and up-to-date dataset. The AI module then pre-processes the collected data by cleaning, normalizing, and integrating it into a consistent format. This step helps eliminate outliers, corrects data inconsistencies, and ensures data quality for accurate analysis. AI algorithms can extract relevant features from the collected data, such as pollutant concentrations, weather conditions, geographical factors, and historical patterns. These features provide crucial inputs for the AQI calculation. The AI module can train a machine learning model using historical air quality data and corresponding AQI values and alert levels. The model learns the complex relationships between different variables and their impact on AQI. Once the model is trained, the AI module can utilize it to predict AQI values in real-time. By feeding the current data inputs into the model, it can generate accurate predictions of AQI levels. The AI module can generate visual representations of AQI data, such as maps, graphs, or charts, to provide intuitive and easily interpretable insights. It can also generate reports or alerts based on predefined thresholds or specific events. The AI module can adapt and improve over time by continuously receiving new data, updating the model, and retraining it periodically. This adaptive learning approach allows the module to enhance its accuracy and performance as it incorporates new information.

In an embodiment, multiple sensors (via crowdsourcing) can be utilized to gather information and thereafter develop “intelligence” on the prediction of air quality. In an embodiment, the system may use image or video data to assess the air quality. In an embodiment, the system may recommend a control option for purifying the air through machine learning and prediction methodologies.

Various models may be used for Air Quality Index (AQI) prediction, depending on the available data and desired accuracy. Regression models, such as linear regression or multiple regression, establish relationships between air quality variables and AQI through statistical analysis. Artificial Neural Networks (ANN) mimic the brain's function and learn complex patterns to predict AQI based on historical data and input variables. Support Vector Machines (SVM) find hyperplanes to separate data based on air quality features and to make AQI predictions. Decision tree models use hierarchical decision rules to predict AQI, while ensemble models combine multiple models to enhance accuracy. These models leverage historical air quality data, meteorological factors, pollutant concentrations, and other relevant variables to generate predictions. By training on vast datasets and analyzing the relationships among input variables and AQI, these models can provide valuable insights into air quality conditions. However, selecting the appropriate model takes into consideration factors such as data availability, computational resources, and desired prediction accuracy. Rigorous evaluation and validation of the chosen model using relevant air quality data are necessary to ensure reliable and accurate AQI predictions, aiding in making informed decisions and taking appropriate control actions to mitigate air pollution.

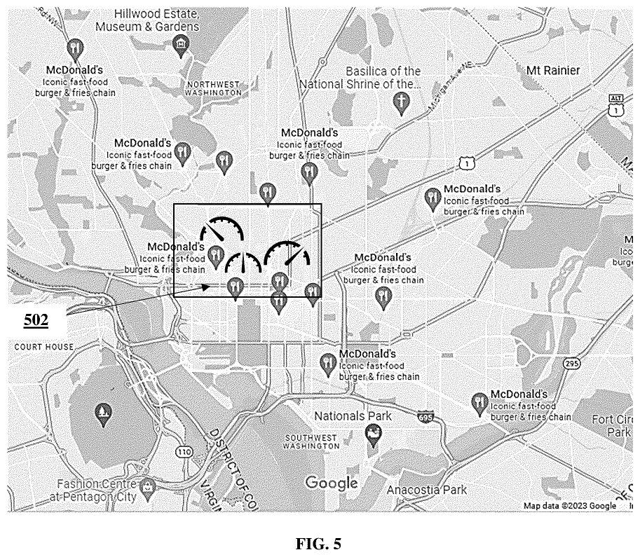

FIG. 5 shows an example display of air quality on a location map according to an embodiment of the system. In an embodiment, when the user searches for a business, for example, Mc Donald's® at an area of interest 502, the geographical map with McDonald's locations may be overlaid with the air quality rating data. In an embodiment, the user may prefer to go to the same business at a location where the air quality rating is better than the other locations. In an embodiment, the system uses crowdsourcing of air quality ratings of indoor spaces available to the public, so that users can validate hygiene of locations that they would like to visit, live, or work.

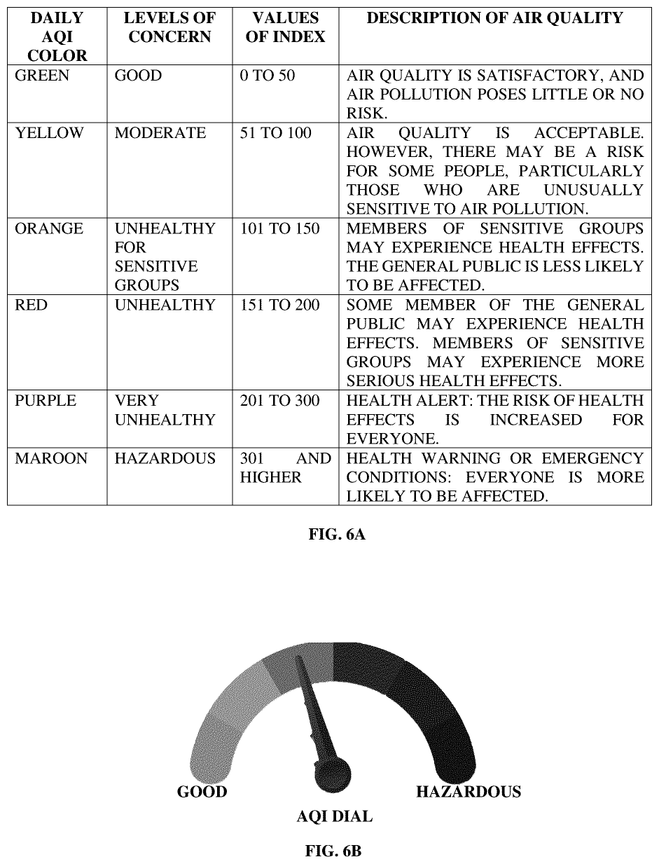

FIG. 6A shows an example of AQI indices and health concerns according to an embodiment of the system. In an embodiment, the classification into groups may be dependent on area to area. In an embodiment, the quantitative values 0-500 may be categorized into qualitative groups like good, moderate, unhealthy for sensitive groups, unhealthy, very unhealthy, and hazardous. In an embodiment, the system correlates the health status of the user while recommending an alternate location. The system may display, on the user device, a health hazard based on the current health status of the user. In an embodiment, the system records the amount of time the user is exposed to various AQI groups and summarizes this for the user.

FIG. 6B shows an example display of quantified AQI using a user-friendly dial icon according to an embodiment of the system. The display may be user friendly and an easy to understand format like a dial where one end is good/healthy, and the other end is unhealthy/hazardous, and the intermediate levels quantified into various colors for easy indication to the user.

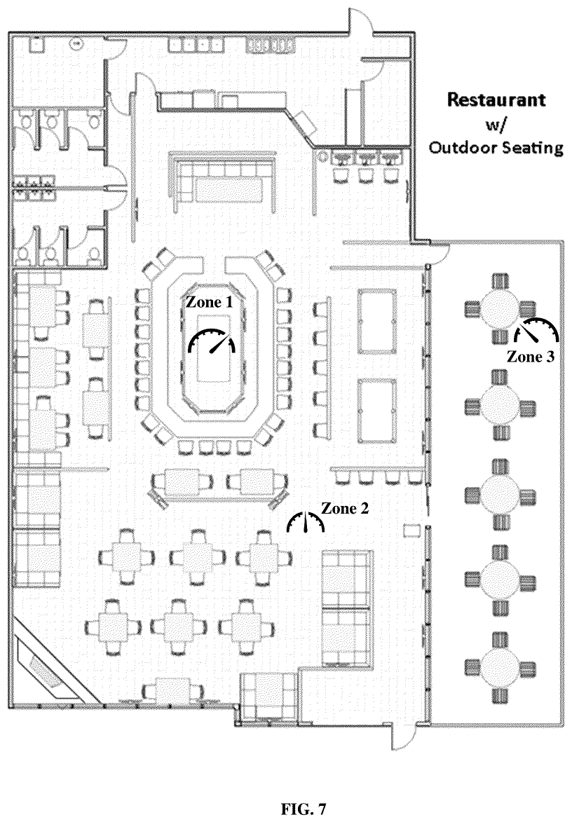

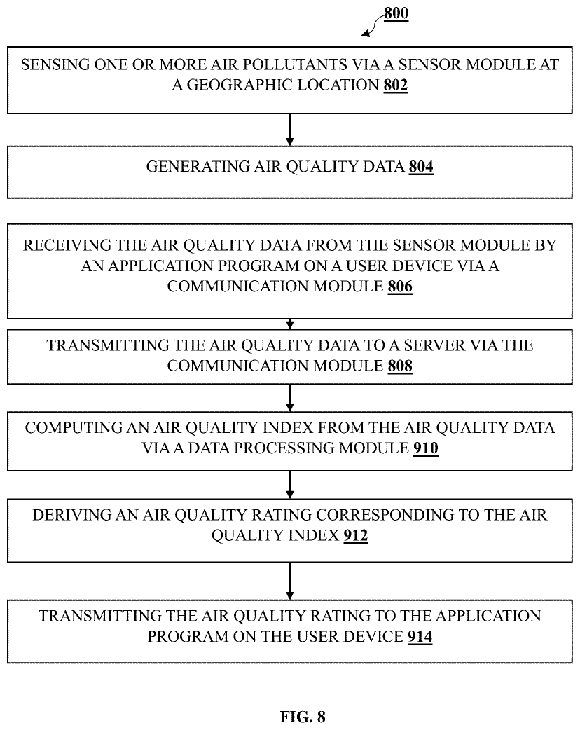

FIG. 7 shows a system suggesting an alternate location in indoor environments based on AQI ratings according to an embodiment. FIG. 7 shows a restaurant having both indoor space and an outdoor space. When a user is at the restaurant, the system may overlay the indoor space plan with the AQ Index or air quality rating such that the user may prefer a location or a spot which is better within the available seating zones. In the figures, the system shows three zones, where Zone 1 may have a bad AQ index compared to Zone 3, and Zone 3 is better than Zone 2 and Zone 1, and is displayed accordingly using the AQ index meter.

According to an embodiment of the system, the system is configured to monitor air quality of an indoor space. According to an embodiment of the system, the indoor space comprises one or more of an office space, a house, a restaurant, a café, a movie theater, a shopping mall, an office building, indoor spaces of a school, indoor spaces of a university, an indoor sports facility, an inside space of a public transportation vehicle, an inside space of a private transport vehicle, an indoor space of a hospital and healthcare facility, an underground parking lot, and an indoor concert venue. According to an embodiment of the system, the system further comprises a control module configured to control a sterilization and circulation unit operable to remedy the air quality of the indoor space. According to an embodiment of the system, the system further comprises a control module configured to control an Ultraviolet device configured to purify air in a vicinity of a user.

According to an embodiment of the system, the system is configured to monitor air quality of an outdoor space. According to an embodiment of the system, the outdoor space comprises one or more of a park, a garden, a beach, a waterfront area, a playground, a sports field, an outdoor sports stadium, a hiking trail, a nature reserve, a public square, a plaza, an open-air market, an outdoor dining area, a patio, a campground, and a rooftop terrace.

Information is easily accessible about the quality of food and services and restaurants by organizations such as Yelp® and Zomato®. One can also view ratings of healthcare professionals on websites. One can even get reviews on handyman services for projects related to home. However, there is no information available about air quality in the businesses and the buildings which are visited in everyday life. Humans spend approximately 90% of time indoors, also, COVID-19 virus-like scenarios or impaired quality is responsible for many health infections and health conditions. The idea is to make available the air quality ratings of indoor spaces to the public so users can validate the hygiene of the locations they would like to visit or work in.

Once the public can access these air quality ratings, it may lead to informed decisions about where they would like to visit indoors, regardless of whether it is a restaurant, auditorium, business, hotel, living space, etc. Also on the flip side, businesses may cite their air quality to assure customers and employees of the hygiene of the facilities and the importance of indoor air quality. Overall, the air quality is vital to health and happiness.

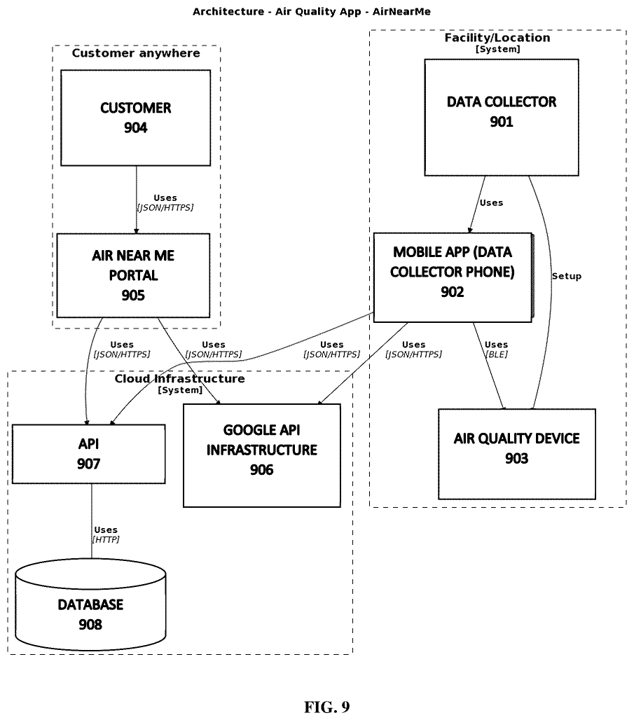

FIG. 8 shows a block diagram of a method of using the system according to an embodiment. According to an embodiment, it is a method 800, comprising: sensing one or more of one or more air pollutants via a sensor module at a geographic location at step 802; generating air quality data at step 804; receiving the air quality data from the sensor module by an application program on a user device via a communication module at step 806; transmitting the air quality data to a server via the communication module at step 808; computing an air quality index from the air quality data via a data processing module at step 910; deriving an air quality rating corresponding to the air quality index at step 912; transmitting the air quality rating to the application program on the user device at step 914; and wherein the air quality data comprises one or more air quality parameters, the geographic location corresponding to the one or more air quality parameters and a time stamp; and wherein the method is configured to implement a crowdsourced sensor system for determining air quality.

According to an embodiment, it is a non-transitory computer-readable medium having stored thereon instructions executable by a computer system to perform operations comprising: receiving an air quality data on a server from an application program on a user device via a communication module; transmitting the air quality data to a server via the communication module; computing an air quality index from the air quality data via a data processing module; deriving an air quality rating corresponding to the air quality index; and transmitting the air quality rating to the application program on the user device; and wherein the air quality data comprises one or more air quality parameters, the geographic location corresponding to the one or more air quality parameters and a time stamp; and wherein the method is configured to implement a crowdsourced sensor system for determining air quality.

The most experienced element on Earth, air, is least monitored in personal and public indoor settings for air quality which can result in the increase of transmissible diseases being spread, as learned during the COVID pandemic. Beyond the transmission of pathogens, air quality could cause health problems like allergies, asthma, cardiovascular diseases, respiratory illnesses, strokes, headaches, nausea, and many more. People with pre-existing lung or cardiovascular conditions could have these conditions worsened and even become critical conditions when experiencing poor indoor air quality for prolonged times. Harmful gases and VOCs, or volatile organic compounds can cause life threatening conditions. Overall, extended exposure to poor indoor air quality has a negative impact on life expectancy, and housings with high indoor humidity can promote mold growth. In workplace settings and schools, poor air quality limits productivity and cognitive functioning. Poor air quality is very uncomfortable.

In an embodiment, business owners who would like to advertise the air quality of the facilities could advertise the air quality information to attract customers. Customers of the businesses, who would like to assess the air quality to determine if it is safe for them to visit the business, can assess the air quality information from the website where the air quality data and ratings are presented. Employees who would like to assess the air quality of their working environments to determine if it is safe can do so. Employers who would like to advertise the air quality to ensure employees that they have safe working conditions, can do so. Property owners may also want to advertise the air quality of their properties to attract candidates and assure people, who are living there actively, that it is safe for them. Tenants could assess the air quality of their living spaces to confirm that it is safe. In addition, many more needs can arise in the future as the air quality service becomes available.

In an embodiment, the system comprises an air sensor, which senses the air parameters including CO2, Total Volatile Organic Compounds, Particulate Matter (PM 2.5 to PM 10), Pathogens, temperature, humidity, air flow. The air sensor is configured to send the data which it senses for the air quality ratings to a smartphone application. Once the data is received, it matches this air quality data with GPS coordinates, for example, with a business location or a restaurant location. The system then inputs the data to a cloud-based web application, which users may access via the internet to view the air quality of the locations they would like to visit. In an embodiment, the user may own the air sensing device. The air sensing device may be coupled with a smartphone to gather location data, whether it being a hotel, a restaurant, a business, etc. In an embodiment, the sensor device, may comprise the GPS sensor and the smartphone, is configured for receiving and transmitting the information from the sensor device and the cloud server. In an embodiment, the data processing unit may be part of the smartphone or part of the cloud network.

While using the air sensing device, the user may sense the air quality of the location. Once this air quality is sensed, the air sensing device transmits this air quality data to a smartphone. When the smartphone receives the air quality data on a smartphone application, it gathers the GPS location data, and it pairs it with the air quality data and time stamps the data. Once this data is matched, it transmits it onto a cloud-based application. On the cloud-based application users and the public can view the air quality data. In an embodiment, the air quality data is paired up with location data and the rating may be displayed when a user searches for a location or a business. The air quality data is a metadata comprising the location, the time it was taken, and the air quality parameters. Metadata refers to data that provides information about other data. It is essentially data about data. Metadata describes various attributes of a particular dataset, document, file, or information resource, offering context and facilitating its discovery, organization, management, and interpretation. From the metadata and AQ Index rating, an overall rating, based on the parameters which are measured via the air sensor device, are computed. AQ Index reading ranges from zero to 300, and zero is the best for air quality, and 300 is the worst.

Sensing, which is a crowdsource component, enables users to go to locations, and then collect the data from the sensors and send them to a smartphone application. The data is then uploaded online so that other people can view or use it. The air quality data is sensed via crowdsourcing using the sensor device or sensor module and makes it available to the common public. The sensor would go with the people and wherever they visit. Sensed data is automatically fed into the mobile application and to the web portal on the cloud, where the information is made available to anybody to figure out what is the air quality of that location.

In the past, restaurants relied on a limited number of rating agencies and food critics to assess their quality. However, with the emergence of platforms like Zomato® and Yelp®, the rating system became more accessible to the general public. This shift allowed anyone and everyone to provide ratings and reviews for any restaurant they visited. This transition to crowd-sourced ratings enabled individuals to share their dining experiences, giving positive ratings for dishes they enjoyed and negative ratings for those they did not. Over the course of 15 years, this crowd-sourced approach has gained popularity, and today it has become a common practice for people to check platforms like Yelp® to review restaurant ratings before deciding where to dine.

The process of trend analysis and prediction involves analyzing past data to make projections for future periods. Corrective action can be taken in two ways, (i) either by seeking an alternative location (ii) by addressing the air quality issue through an interface with the Heating, Ventilation, and Air Conditioning (HVAC). These two actions are applicable for outdoor as well as indoor remedies. For example, if the user is in a burger joint with poor air quality, the system can suggest an alternative burger joint located one mile away. This provides a simple solution for customers to consider, ultimately addressing the issue at hand.

In another example, if there is a Burger King® at location 1 and the system detects poor air quality at location 1, it can recommend another Burger King® near to location 1 with better air quality or with an air quality that is suitable for the user based on his health and preferences. This recommendation is based on the information in the database. The system distinguishes between outdoor and indoor air quality monitoring, with outdoor monitoring being more established and indoor more controllable.

Indoor air quality is a significant concern that demands attention. For instance, when searching for an apartment as a tenant, it is crucial to consider indoor air quality. Although this may not be as relevant in certain areas, it becomes apparent in situations like staying at expensive hotels within a renowned chain. Upon entering such hotels, one might notice a slightly musty or stale smell, indicating insufficient air circulation. This issue is particularly prominent due to prolonged exposure to poorly circulated air over several months, in which case the sensor may detect such a scenario and advise for air circulation and ventilation activation. On the other hand, the system may recommend another location.

For instance, for implementing corrective action, the system refers back to the database and analyzes GPS data to identify similar businesses within a two or three square mile radius. If there is a suitable alternative available, a signal or trigger is sent. This process can be adequately described and implemented without major difficulty. In the corrective action, where the HVAC system has to be controlled, an Application Program Interface (API) that interfaces with the control unit can be exercised.

An Example calculation of Air Quality Index from Sensor measurements: An example is provided on how the Air Quality Index (AQI) may be calculated based on the concentrations of three common pollutants PM2.5, NO2, and O3 as presented herein. It may be understood that the calculations in this example are simplified and are presented for illustrative purposes only. In embodiments, the AQI is calculated from all the air pollutants detected and measured.

Measure Pollutant Concentrations via the sensor module:
i. PM2.5 concentration: 25 μg/m³
ii. NO2 concentration: 40 ppb (parts per billion)
iii. O3 concentration: 60 ppb

Calculate Pollutant Index Values:

PM2.5 Index Value: Suppose the PM2.5 index has the following breakpoints:
i. 0-12 μg/m³: Good (AQI=0-50)
ii. 12.1-35.4 μg/m³: Moderate (AQI=51-100)
iii. 35.5-55.4 μg/m³: Unhealthy for sensitive groups (AQI=101-150)
iv. 55.5-150.4 μg/m³: Unhealthy (AQI=151-200)
v. 150.5-250.4 μg/m³: Very unhealthy (AQI=201-300)
vi. 250.5+μg/m³: Hazardous (AQI=301+)

Since the PM2.5 concentration is 25 μg/m³, it is categorized within the “Good” range, so the PM2.5 index value is 25.

NO2 Index Value: Suppose the NO2 index has the following breakpoints:
i. 0-53 ppb: Good (AQI=0-50)
ii. 53.1-100 ppb: Moderate (AQI=51-100)
iii. 100.1-360 ppb: Unhealthy for sensitive groups (AQI=101-150)
iv. 360.1-649 ppb: Unhealthy (AQI=151-200)
v. 649.1-1249 ppb: Very unhealthy (AQI=201-300)
vi. 1249+ ppb: Hazardous (AQI=301+)

Since the NO2 concentration is 40 ppb, it falls within the “Good” range, so the NO2 index value is 40.

O3 Index Value: Suppose the O3 index has the following breakpoints:
i. 0-54 ppb: Good (AQI=0-50)
ii. 54.1-70 ppb: Moderate (AQI=51-100)
iii. 70.1-85 ppb: Unhealthy for sensitive groups (AQI=101-150)
iv. 85.1-105 ppb: Unhealthy (AQI=151-200)
v. 105.1-200 ppb: Very unhealthy (AQI=201-300)
vi. 200+ ppb: Hazardous (AQI=301+)

Since the O3 concentration is 60 ppb, it falls within the “Moderate” range, so the O3 index value is 60.

Determine Dominant Pollutant:

In this example, the O3 concentration has the highest index value, making it the dominant pollutant.

Overall AQI Calculation:

The AQI value is derived from the index value of the dominant pollutant. In this case, the AQI value is 60, corresponding to the “Moderate” air quality category.

The actual calculations and breakpoints for AQI may vary based on specific regional or national standards. This example is simplified and provided for illustrative purposes. The AI module considers complex relations among these variables to compute and predict an AQI index.

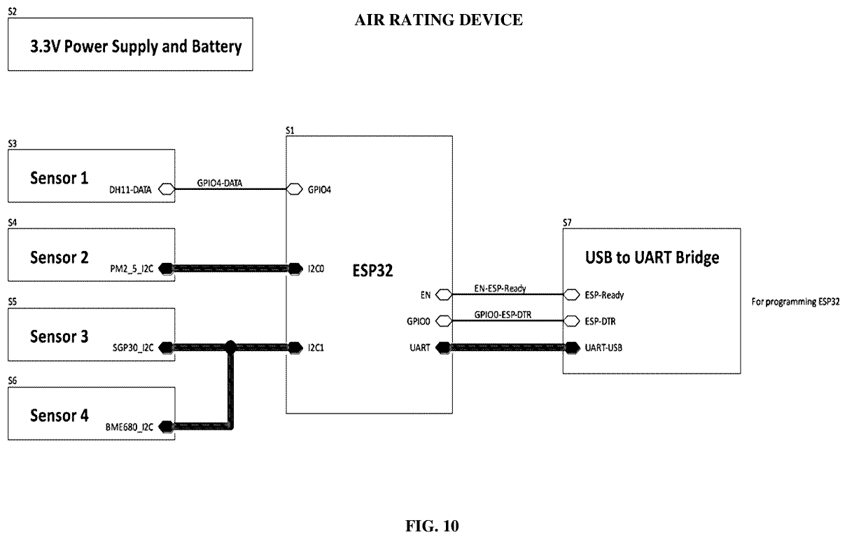

FIG. 9 shows the architecture of the system according to an embodiment. The architecture comprises an air quality data collector 901, who collects data with air quality device and mobile application. Air quality data collector is any person who has the air sensing device and is using that to sense the air quality data in a location of a facility around them. For example, if the person is at McDonald's®, and turns the air sensing system on, and then the person is sensing the data. The person will be using the air quality device to sense the data. Once the data is sensed by the air quality device, it connects to the smartphone mobile application 902, which is data collector phone via Bluetooth®, and then uploads that collected air quality data to the phone. The mobile application 902 connects the air quality device 903 and helps ingest data to cloud. The Air quality device 903, has the air quality sensors and connects to the smartphone, the smartphone uploads the data using the smartphone application to the Cloud network.

Once the Air Quality data is uploaded to the cloud back-end infrastructure via the sensor, any user using a web portal, including a user who does not have an air quality sensor, but are interested in checking the air quality data of location, that user, as shown in FIG. 9 as the air quality data consumer 904, can view and download the data. Air quality data consumers may not upload the data themselves but view it. Air quality data customer is a customer of AirNearMe® application. In an embodiment, Air quality data collector is also the Air quality data customer. These customers use the air quality web portal (AirNearMe® Portal) 905, which provides all the Air quality data for the searches or current location. It is interfaced with the Google Maps API. Customers/users will be able to search up the specific location, current location, or address and the data from the cloud would be pulled out corresponding to that location.

The system uses Cloud infrastructure, which refers to the components required to support cloud computing services. It encompasses a combination of hardware, software, networking, and services that enable the storage, processing, and access of data and applications over the internet. At its core, cloud infrastructure relies on servers, both physical and virtual, which provide the necessary computing power for running software (Ex. Application software) and hosting services. Additionally, cloud infrastructure offers scalable and flexible storage solutions, allowing organizations to store and manage their data (Ex. Air Quality Data) efficiently. Networking is used for connecting various components within the cloud infrastructure, facilitating seamless communication and data transfer. Virtualization technology enables the creation of virtual machines or containers, maximizing resource utilization and enabling easy scalability. Management and orchestration tools streamline the provisioning, configuration, and monitoring of cloud resources. Security measures are also an integral part of cloud infrastructure, ensuring data protection, identity management, and defense against cyber threats. Cloud infrastructure provides a diverse range of services that can be easily provisioned, scaled, and billed based on usage, empowering businesses with agility and cost-effectiveness.

In an embodiment, a data collector or user will have a smartphone and the sensor module. In another embodiment, the sensor module may be integrated with the smartphone. The sensor module transmits the air quality data to the smartphone and the smartphone captures the GPS data of its location.

The Cloud infrastructure is on Amazon Web Services (AWS), which is a comprehensive cloud computing platform offered by Amazon®, providing a wide range of services and infrastructure for building, deploying, and managing applications in the cloud. Within the AWS ecosystem, Transmission Control Protocol (TCP) is the protocol used for reliable communication. TCP is a connection-oriented protocol operating at the transport layer of the Internet Protocol Suite. It ensures reliable and ordered data transfer between devices over a network. In the context of AWS, TCP is extensively utilized for various purposes. Firstly, it facilitates communication between instances within an AWS Virtual Private Cloud (VPC), enabling secure and dependable interaction between different components. Secondly, AWS employs TCP in its Elastic Load Balancing (ELB) service, which distributes incoming traffic across multiple instances or containers, ensuring balanced workloads and efficient resource utilization. Furthermore, TCP is instrumental in network security within AWS, as it forms the basis for security groups and firewalls that control inbound and outbound traffic to instances, allowing granular control over network access. Lastly, TCP serves as the underlying transport protocol for numerous application-layer protocols, such as Hypertext Transfer Protocol (HTTP) and Hypertext Transfer Protocol Secure (HTTPS), enabling the seamless functioning of web applications, Application Program Interface (APIs), and other services hosted on AWS. TCP is a vital protocol within AWS, providing reliable data transfer, enabling inter-instance communication, facilitating load balancing, ensuring network security, and supporting various application-layer protocols. Its incorporation within the AWS infrastructure strengthens the robustness, scalability, and security of applications and services deployed on the platform, contributing to the overall effectiveness of the AWS cloud computing environment. The geographic positioning data may come from the smartphone having location services.

Google API infrastructure 906 forms the foundation for the APIs offered by Google. It helps to integrate the Google API with necessary details for ease of mapping the location data. At its core, the infrastructure comprises an API Gateway that serves as a centralized entry point for developers to access and interact with Google's APIs. It manages authentication, authorization, and access control, ensuring that only authorized users and applications can make API requests. Supporting the API Gateway is Google's expansive service infrastructure, comprises a distributed network of data centers and servers. This infrastructure is designed to handle the massive scale of Google's services, providing scalability, reliability, and high performance. To efficiently route API requests, Google utilizes service proxies that handle load balancing, traffic routing, and fault tolerance. These proxies ensure that requests are directed to the appropriate backend services, optimizing response times and availability. Additionally, Google's API infrastructure includes robust authentication and authorization mechanisms, allowing developers to secure their API access using API keys or OAuth 2.0. Google API infrastructure is a robust and sophisticated system that enables to leverage the extensive capabilities of Google's services through well-designed APIs which is utilized in developing the system for Air Quality. Further, API's 907 provides information on location, air quality, date, time etc. The system comprises database 908 which stores the air quality data along with location, date, and time.

FIG. 10 shows the Sensor module architecture according to an embodiment. It comprises various sensors depicted as Sensor 1, Sensor 2, Sensor 3, Sensor 4 etc. Though the figure shows 4 sensors, there is no limitation on the number of sensors or the types of sensors that may be accommodated and integrated into the system.

Sensor 1 is a DHT11 sensor which is a digital temperature and humidity sensor module. It provides temperature and humidity measurements. The DHT11 sensor utilizes a capacitive humidity sensor and a thermistor to measure the relative humidity and temperature, respectively. It has a single-wire digital interface, making it easy to connect to microcontrollers or other devices. The sensor is capable of measuring temperatures ranging from 0 to 50 degrees Celsius (32 to 122 degrees Fahrenheit) with an accuracy of ±2 degrees Celsius (±3.6 degrees Fahrenheit). It can measure relative humidity within a range of 20% to 80% with an accuracy of ±5%. In an embodiment, the DHT11 sensor can be replaced with any other advanced sensors available in the market for more accuracy. The DHT11 sensor is used for environmental monitoring. For higher accuracy or more precise measurements, other sensors like the DHT22 or SHT series sensors such as SHT71 may be used in place of DH11.

Sensor 2 is a PM2.5 sensor, which is an air quality sensor specifically designed to measure the concentration of particulate matter (PM) with a diameter of 2.5 micrometers or smaller in the air. PM2.5 refers to fine particles suspended in the air that are smaller than 2.5 micrometers, which can be harmful to human health when inhaled. The PM2.5 sensor utilizes various technologies, including optical, laser, or electrochemical methods, to detect and quantify the concentration of these fine particles. It provides real-time measurements of PM2.5 levels in the surrounding environment, allowing users to monitor air quality and take appropriate actions to mitigate potential health risks. The data obtained from this sensor is used for assessing air pollution levels, identifying sources of pollution, and implementing measures to improve air quality. In an embodiment, it is accompanied with sensors measuring other pollutants, such as carbon dioxide (CO2), volatile organic compounds (VOCs), and nitrogen dioxide (NO2), for comprehensive air quality monitoring.

Sensor 3 is a SGP30 sensor which is a gas sensor module comprising VOC and Carbon Dioxide Sensor designed to detect and measure indoor air quality for VOC and Carbon Dioxide. The acronym SGP stands for Sensirion® Gas Platform, indicating that it is a product developed by Sensirion®, a Swiss sensor manufacturer. The SGP30 sensor specifically focuses on detecting volatile organic compounds (VOCs) and measuring the concentration of carbon dioxide (CO2) in the air. The SGP30 sensor incorporates a metal oxide semiconductor (MOx) sensing element and uses an innovative multi-pixel technology. It operates based on the principle of resistance changes in the sensing element when exposed to different gases. The sensor is calibrated for indoor air quality applications and provides reliable and accurate measurements. The SGP30 sensor communicates with microcontrollers or other devices using the I2C (Inter-Integrated Circuit) protocol, allowing for easy integration into various electronic systems. It offers low power consumption, making it suitable for battery-operated devices and IoT (Internet of Things) applications. With the SGP30 sensor, the system monitors and assesses the air quality in indoor environments, enabling them to take appropriate measures for ventilation, air purification, and overall comfort. The sensor's ability to detect VOCs and Carbon Di Oxide (CO₂) makes it useful in applications such as smart homes, air purifiers, air quality monitors, and HVAC (Heating, Ventilation, and Air Conditioning) systems.

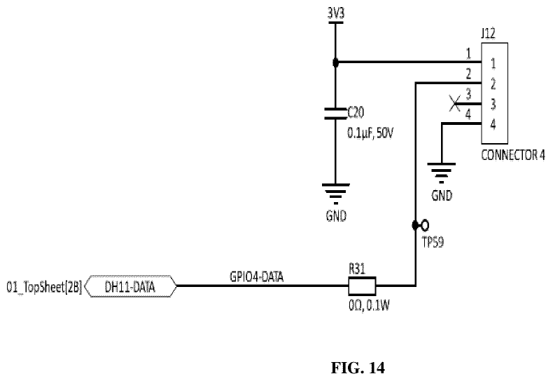

Sensor 4 is a BME680 sensor, developed by Bosch® Sensortec, is a versatile environmental sensor module that integrates four sensors into a single package and is a VOC, Temperature, Humidity Sensor. It is designed to measure temperature, humidity, air pressure, and volatile organic compounds (VOCs) in the surrounding environment. The temperature sensor provides accurate temperature measurements, while the humidity sensor measures relative humidity with good precision. The built-in pressure sensor allows for atmospheric pressure measurement and estimation of altitude. Additionally, the BME680 includes a gas sensor capable of detecting and measuring VOCs, providing insights into air quality and environmental changes. This sensor is an alternate sensor that could be used in place of the DHT11 sensor as shown in FIG. 14 and senses VOCs. In an embodiment, the data from both the sensors is combined using sensor fusion methods. Sensor fusion is the process of combining sensor data or data derived from disparate sources such that the resulting information has less uncertainty than would be possible when these sources were used individually.

Various sensors are connected to a microcontroller. ESP32 is a popular and widely used microcontroller and Wi-Fi/Bluetooth module developed by Espressif Systems®. It is part of the ESP series of microcontrollers and is known for its powerful features, versatility, and low cost. The ESP32 is based on a dual-core Xtensa LX6 microprocessor, which offers high computing power and clock speeds up to 240 MHz. One of the key features of the ESP32 is its built-in Wi-Fi and Bluetooth capabilities, allowing for wireless communication and connectivity. It supports various Wi-Fi standards, including 802.11 b/g/n, and offers both client and access point modes. The integrated Bluetooth module supports classic Bluetooth as well as Bluetooth Low Energy (BLE), enabling easy integration. The ESP32 provides a wide range of peripherals and interfaces, including GPIO (General Purpose Input/Output) pins, SPI (Serial Peripheral Interface), I2C (Inter-Integrated Circuit), UART (Universal Asynchronous Receiver-Transmitter), and more. This makes it highly versatile and suitable for a variety of applications, such as Internet of Things (IoT) devices, home automation, robotics, sensor networks, and industrial applications. Development for the ESP32 is done using various programming languages, including Arduino, MicroPython, and the Espressif IoT Development Framework (ESP-IDF), which provides a low-level programming interface for the Air Quality application.

A USB to UART bridge is a type of hardware device or integrated circuit (IC) that enables communication between a computer's USB port and a microcontroller or other UART-enabled device. It acts as a bridge, converting the USB signals from the computer into UART signals that the microcontroller can understand and vice versa. The USB to UART bridge typically comprises a USB controller, UART controller, and associated circuitry. It allows the microcontroller to be connected to the computer via USB, providing a convenient way to program, debug, and exchange data with the microcontroller. The USB to UART bridge IC translates the USB protocol into UART serial communication, which follows the standard asynchronous serial communication format with start and stop bits. It supports various baud rates, data formats, and flow control options, allowing for flexible and reliable communication between the computer and the microcontroller. In an embodiment, a microcontroller development board, such as the Arduino, ESP8266, and ESP32, which incorporate a USB to UART bridge on-board may be chosen. This bridge allows users to connect the development board to a computer for programming, uploading firmware, and serial communication. USB to UART bridges provide a straightforward way to establish communication between a computer and a UART-based device using the USB interface.

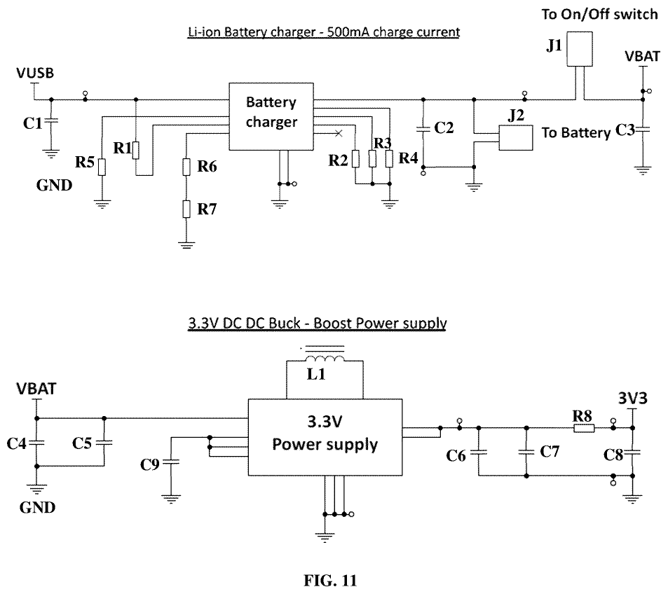

FIG. 11 shows Li-Ion Battery charger and Boost power supply circuit diagram according to an embodiment. The Li-Ion battery charger circuit is composed of various components that work together to charge the Li-Ion battery safely and efficiently. It starts with a power source, which can be AC mains or a DC power supply. If AC mains is used, a transformer steps down the voltage, followed by a rectifier circuit to convert AC to DC. A filter capacitor smooths the rectified DC voltage. To ensure safety, a current-limiting resistor or fuse is included. The charging process is managed by a constant current (CC) and constant voltage (CV) charging circuit, with a voltage reference element to set the charging voltage. A charging controller or IC regulates the charging process, while a power transistor or switch controls the charging current. A feedback mechanism monitors the battery voltage and adjusts the charging current accordingly. Protection circuits prevent overcharging, overcurrent, and short circuits, ensuring the battery is charged safely and efficiently. The 3.3V DC-DC buck-boost power supply circuit enables the conversion of an input voltage (which can be higher or lower than 3.3V) to a stable 3.3V output. The circuit typically incorporates a dedicated buck-boost converter IC that is specifically designed for the desired voltage conversion. The input and output capacitors serve to filter and stabilize the voltage. An inductor is included to store and release energy during the switching process. Control circuitry regulates the switching of the converter, while feedback mechanisms monitor the output voltage and adjust the switching accordingly. Optional protection circuits such as overvoltage, overcurrent, and thermal protection may be added for enhanced safety. By adjusting the duty cycle of the switching transistor, the buck-boost converter can step up (boost) or step down (buck) the input voltage as necessary to maintain a stable 3.3V output, providing a reliable power supply for various applications. In an embodiment, the circuit design may involve additional components and considerations based on specific requirements and safety standards.

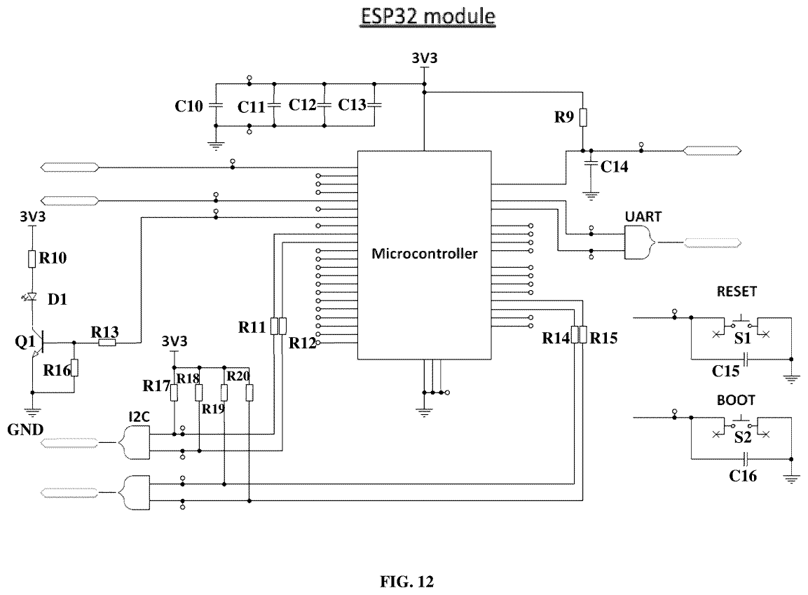

FIG. 12 shows a circuit diagram for ESP32 module according to an embodiment. The ESP32 module circuit is composed of various components that work together to enable the functioning of the ESP32 microcontroller and its associated features. At the heart of the circuit is the ESP32 microcontroller, which may include a powerful dual-core Xtensa LX6 processor, memory, and a range of peripherals and connectivity interfaces. It executes the program instructions, manages input/output operations, and controls the overall operation of the module. The flash memory provides non-volatile storage for firmware, programs, and data. Integrated Wi-Fi and Bluetooth modules allow for wireless communication and connectivity with other devices and networks. A crystal oscillator generates precise clock signals for synchronization and timing. A voltage regulator ensures stable and reliable power supply to the module. GPIO pins provide versatile interfaces for connecting external devices and peripherals, supporting digital input/output, analog input, and specialized interfaces such as UART, SPI, and I2C. An integrated antenna facilitates wireless signal transmission over Wi-Fi and Bluetooth. The USB interface enables connection to a computer or other devices for programming, debugging, and data transfer. The ESP32 module circuit integrates processing power, connectivity, and flexibility to offer a robust solution for embedded systems and IoT applications.

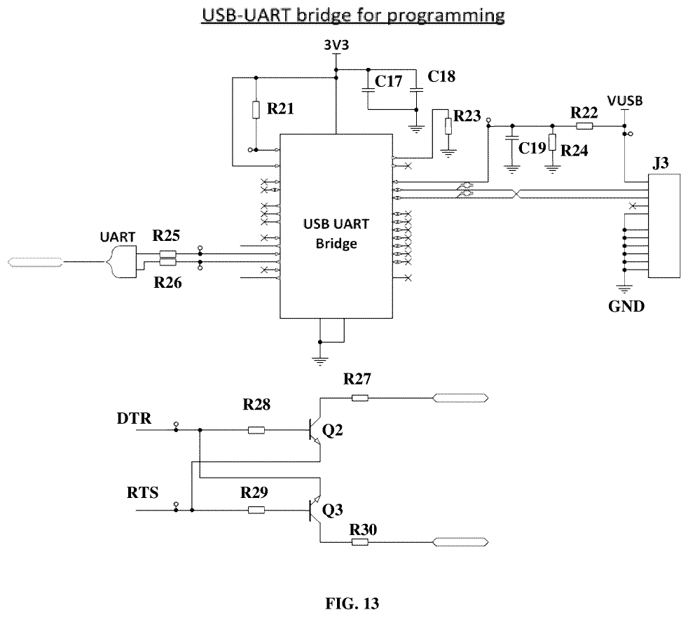

FIG. 13 shows Universal Serial Bus-Universal Asynchronous Receiver-Transmitter (USB-UART) bridge circuit diagram for programming according to an embodiment. The USB-UART bridge circuit diagram for programming establishes a communication link between a computer and a microcontroller's UART interface. The circuit comprises a USB connector, a USB-UART bridge IC or module, and the microcontroller with its UART interface. The USB connector serves as the interface to connect the circuit to a computer or other USB-enabled devices. The USB-UART bridge, typically implemented with a dedicated IC or module, acts as a translator between the USB protocol used by the computer and the UART protocol used by the microcontroller. It converts USB signals to UART signals and vice versa. The USB-UART bridge IC/module is connected to the USB connector, enabling data transfer between the computer and the microcontroller. The microcontroller, equipped with its UART interface, features transmit (TX) and receive (RX) pins. The TX pin of the USB-UART bridge is connected to the RX pin of the microcontroller, allowing data transmission from the computer to the microcontroller. Similarly, the RX pin of the USB-UART bridge is connected to the TX pin of the microcontroller, enabling data transmission from the microcontroller to the computer. This bidirectional communication enables programming and debugging of the microcontroller. By establishing the USB-UART bridge circuit, the microcontroller can communicate with the computer for programming purposes. It enables the transfer of program code, configuration data, and other information between the computer and the microcontroller. This allows firmware updates, debugging operations, and other development tasks to be performed conveniently, as the USB interface provides a standardized and widely compatible means of connection.

FIG. 14 shows digital temperature humidity sensor circuit diagram according to an embodiment. The DH11 sensor circuit diagram comprises the DH11 sensor module and the microcontroller to which it is connected. The DH11 sensor module circuit comprises a DH11 sensor module containing a temperature and humidity sensor chip that measures the ambient temperature and relative humidity in the surrounding environment. A microcontroller, such as Arduino or ESP8266, serves as the central processing unit of the circuit. It interfaces with the DH11 sensor module to receive temperature and humidity data for further processing or display. A pull-up resistor is connected between the data pin of the DH11 sensor module and the supply voltage (VCC) to ensure stable data communication. The DH11 sensor module requires a power supply typically ranging from 3.3V to 5V, depending on the specifications of the module and the microcontroller used. In operation, the microcontroller communicates with the DH11 sensor module using a specific communication protocol, such as the 1-Wire or the proprietary protocol used by the DH11 sensor. The microcontroller sends a command to the DH11 sensor module to initiate a temperature and humidity measurement. The sensor module then measures the temperature and humidity values using its internal sensing element. The measured data is sent back to the microcontroller through the data pin of the DH11 sensor module. The microcontroller reads the data from the DH11 sensor module and processes it according to the desired application. It can display the temperature and humidity values on an LCD screen, store them in memory for later analysis, or transmit them to other devices through communication interfaces like UART or Wi-Fi. The pull-up resistor ensures proper voltage levels on the data line, preventing signal distortion and ensuring reliable communication between the DH11 sensor module and the microcontroller. The microcontroller serves as the control and processing unit, receiving and interpreting the sensor data to perform various actions or provide information to users.

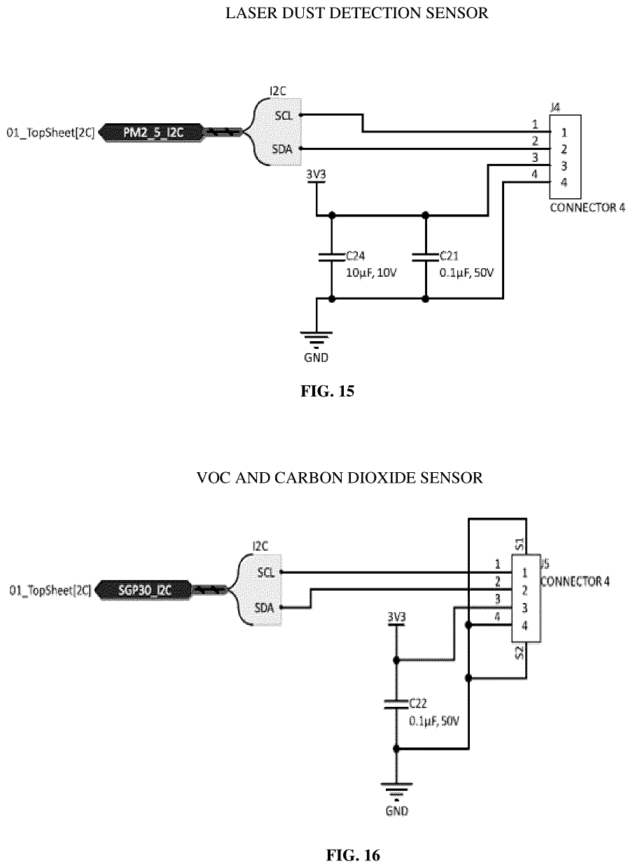

FIG. 15 shows a laser dust detection sensor circuit diagram according to an embodiment. The PM2.5 sensor circuit diagram comprises the PM2.5 sensor module and the accompanying components for interfacing with a microcontroller. The PM2.5 sensor module comprises the sensor element responsible for detecting and measuring the concentration of fine particulate matter (PM) with a diameter of 2.5 micrometers or smaller in the air. This sensor may utilize optical, laser, or electrochemical principles for accurate measurement. A microcontroller, such as Arduino or ESP8266, serves as the main control unit of the circuit. It interfaces with the PM2.5 sensor module to receive and process the measured data for further analysis or display. The PM2.5 sensor module typically requires a power supply ranging from 3.3V to 5V, depending on the specific module used. This voltage is supplied by a microcontroller or an external power source. A pull-up resistor is connected between the data pin of the PM2.5 sensor module and the supply voltage (VCC) to stabilize the data communication line. The PM2.5 sensor module may utilize a specific communication protocol, such as UART or I2C, to transmit the measured data to the microcontroller. The microcontroller should be configured to communicate with the sensor module using the appropriate protocol. In operation, the microcontroller communicates with the PM2.5 sensor module to request and receive the measured PM2.5 data. Depending on the sensor module's design, the microcontroller may send commands or queries to initiate the measurement or configure certain parameters. The PM2.5 sensor module uses its internal sensing element to detect and measure the concentration of fine particulate matter in the air. The sensor converts the measured data into an electrical signal, which is transmitted to the microcontroller through the communication interface. The microcontroller receives the PM2.5 data from the sensor module and can process it according to the application requirements. It may display the PM2.5 readings on an LCD screen, store the data in memory for further analysis, or transmit the information to other devices or systems through various communication interfaces like UART, Wi-Fi, or IoT protocols. The pull-up resistor ensures stable voltage levels on the data line, preventing signal distortion and facilitating reliable communication between the PM2.5 sensor module and the microcontroller. The PM2.5 sensor module circuit enables the detection and measurement of fine particulate matter in the air for air quality monitoring, indoor environmental monitoring, and pollution control. The microcontroller acts as the control center, facilitating communication with the sensor module and processing the PM2.5 data for various purposes.

FIG. 16 shows a carbon dioxide and VOC sensor circuit diagram according to an embodiment. The gas sensor SGP30 circuit diagram comprises the SGP30 gas sensor module and the associated components required for interfacing it with a microcontroller. The SGP30 module comprises the gas sensor element responsible for detecting and measuring volatile organic compounds (VOCs) and the concentration of carbon dioxide (CO2) in the surrounding air. According to an embodiment, the sensor utilizes metal oxide semiconductor (MOx) technology for accurate gas detection. A microcontroller, such as Arduino or ESP8266, serves as the main control unit of the circuit. It interfaces with the SGP30 module to request gas measurements and process the data for further analysis or display. The SGP30 module typically requires a power supply voltage of around 3.3V to 5V, depending on the specific module used. This voltage is provided by the microcontroller or an external power source. The SDA (Serial Data) and SCL (Serial Clock) lines of the SGP30 module are connected to the microcontroller with pull-up resistors to stabilize the data communication lines. The SGP30 module employs the I2C (Inter-Integrated Circuit) protocol for communication with the microcontroller. The microcontroller needs to configure its I2C interface to communicate with the SGP30 module effectively. In operation, the microcontroller communicates with the SGP30 module using the I2C protocol to initiate gas measurements and retrieve the measured VOC and CO2 data. The microcontroller sends commands or queries to the SGP30 module to trigger gas sensing cycles and obtain the gas concentration values. The SGP30 module's gas sensor element detects the VOCs and CO2 present in the air. It converts the measured gas concentrations into electrical signals, which are transmitted to the microcontroller through the I2C communication interface. The microcontroller receives the gas concentration data from the SGP30 module and can process it according to the application requirements. It may display the gas concentrations on an LCD screen, store the data in memory for further analysis, or transmit the information to other devices or systems through various communication interfaces. The pull-up resistors ensure stable voltage levels on the data lines, preventing signal distortion and enabling reliable communication between the SGP30 module and the microcontroller. The SGP30 gas sensor module circuit enables the detection and measurement of VOCs and CO2 in the air, providing valuable data for indoor air quality monitoring, environmental sensing, and smart home automation. The microcontroller serves as the central processing unit, facilitating communication with the sensor module and processing the gas concentration data for various purposes.

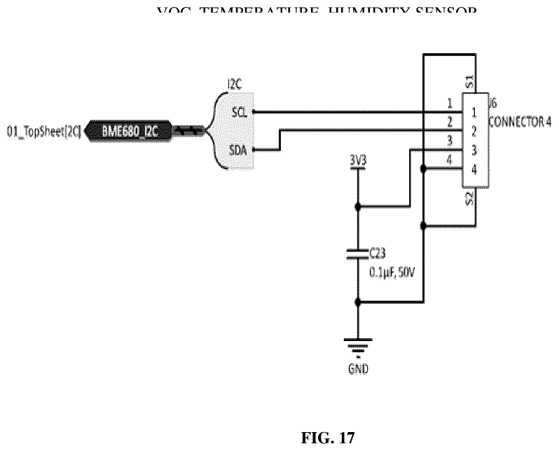

FIG. 17 shows a VOC, temperature, and humidity sensor circuit diagram according to an embodiment. In an embodiment, the air quality sensor is BME680. The BME680 sensor circuit diagram comprises the BME680 sensor module, and the accompanying components needed for interfacing with a microcontroller. The BME680 module contains the environmental sensor chip responsible for measuring temperature, humidity, air pressure, and volatile organic compounds (VOCs) in the surrounding environment. The sensor chip utilizes various technologies, such as temperature and humidity sensors, a pressure sensor, and a gas sensor, to provide accurate measurements. A microcontroller, such as Arduino or ESP8266, acts as the main control unit of the circuit. It interfaces with the BME680 sensor module to receive the environmental data and process it for further analysis or display. The BME680 module typically requires a power supply voltage ranging from 3.3V to 5V, depending on the specific module used. This voltage can be provided by the microcontroller or an external power source. The I2C (Inter-Integrated Circuit) data lines, SDA (Serial Data) and SCL (Serial Clock), of the BME680 module are connected to the microcontroller with pull-up resistors to stabilize the data communication lines. The BME680 module employs the I2C protocol for communication with the microcontroller. The microcontroller needs to configure its I2C interface to establish proper communication with the BME680 module. In operation, the microcontroller communicates with the BME680 module using the I2C protocol to request the environmental data. The microcontroller sends commands or queries to the BME680 module to initiate temperature, humidity, pressure, and gas measurements. The BME680 module's environmental sensor chip detects and measures the temperature, humidity, air pressure, and VOCs in the surrounding environment. The sensor chip converts the measured data into electrical signals, which are transmitted to the microcontroller through the I2C communication interface. The microcontroller receives the environmental data from the BME680 module and can process it according to the application requirements. It may display the temperature, humidity, pressure, and VOC readings on an LCD screen, store the data in memory for further analysis, or transmit the information to other devices or systems through various communication interfaces like UART, Wi-Fi, or IoT protocols. The pull-up resistors ensure stable voltage levels on the data lines, preventing signal distortion and facilitating reliable communication between the BME680 module and the microcontroller The BME680 sensor module circuit enables the measurement of temperature, humidity, air pressure, and VOCs in the environment, for indoor air quality assessment, and environmental sensing. The microcontroller acts as the control center, facilitating communication with the sensor module and processing the environmental data for various purposes. This sensor is an alternate sensor that could be used in place of the DHT11 sensor as shown in FIG. 14 and senses VOCs. In an embodiment, the data from both the sensors is combined using sensor fusion methods. Sensor fusion is the process of combining sensor data or data derived from disparate sources such that the resulting information has less uncertainty than would be possible when these sources were used individually.

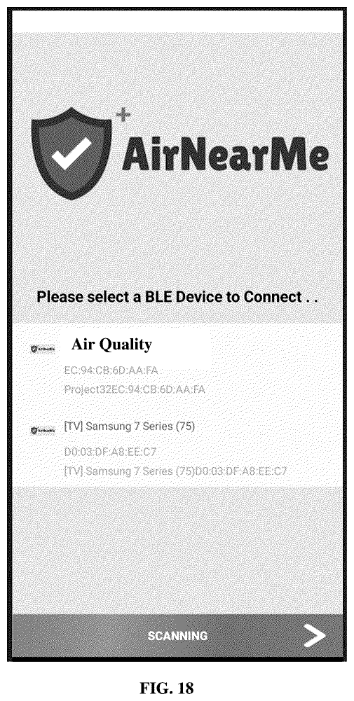

FIG. 18 shows a smartphone application program interface according to an embodiment. The interface is enabled for connecting with Bluetooth Low Energy (BLE) devices, which is a wireless communication technology designed for short-range communication between devices. A BLE device refers to any device that utilizes Bluetooth Low Energy technology for communication purposes. BLE devices establish a connection using Bluetooth radio waves in the 2.4 GHz frequency band. They use a master-slave architecture, where one device acts as the master and initiates the connection, while other devices act as slaves and respond to the master's requests. BLE technology is optimized for short bursts of data transmission, making it suitable for applications that require periodic updates or low data transfer rates. It is particularly efficient in scenarios where power consumption is a critical consideration, such as wearable devices that need to operate for extended periods without frequent battery replacement or charging. BLE devices are compatible with smartphones and other Bluetooth-enabled devices. In an embodiment, the BLE device is the Air Quality Sensor module to which the application program can connect and collect the data and/or provide or access the Air Quality Data which was available on the cloud.

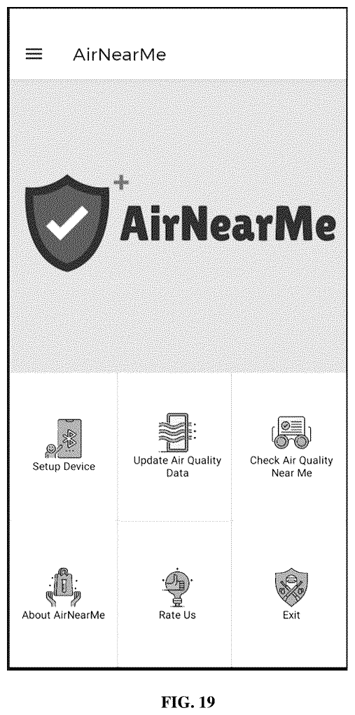

FIG. 19 shows a smartphone application menu interface according to an embodiment. It may comprise options for setting up of the device (which may be an air quality sensor), updating air quality data, checking air quality at a location, more information under About air near me, a rating collection, and exiting the menu.

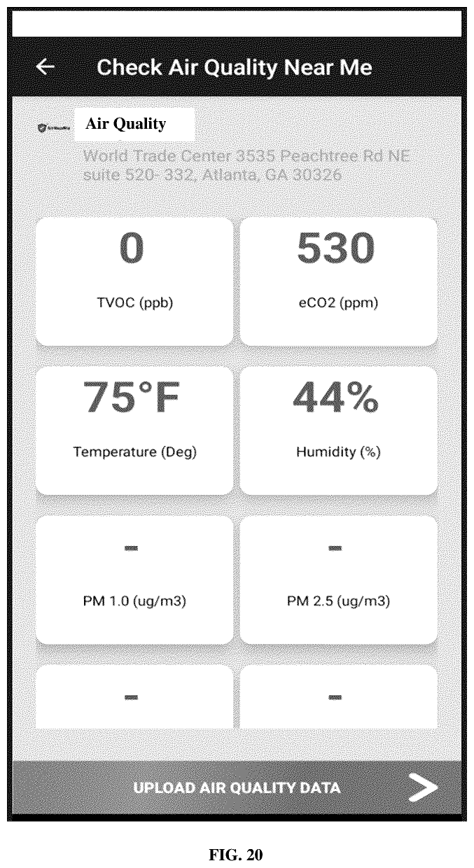

FIG. 20 shows a smartphone application displaying Air Quality parameters sensed by the sensor module according to an embodiment. It displays Air Quality parameters such as Total Volatile Organic Compounds (TVOC), Carbon Dioxide, Temperature, Humidity, Particulate Matter, Pathogens, etc. Users can upload the data using the option providing for upload data.

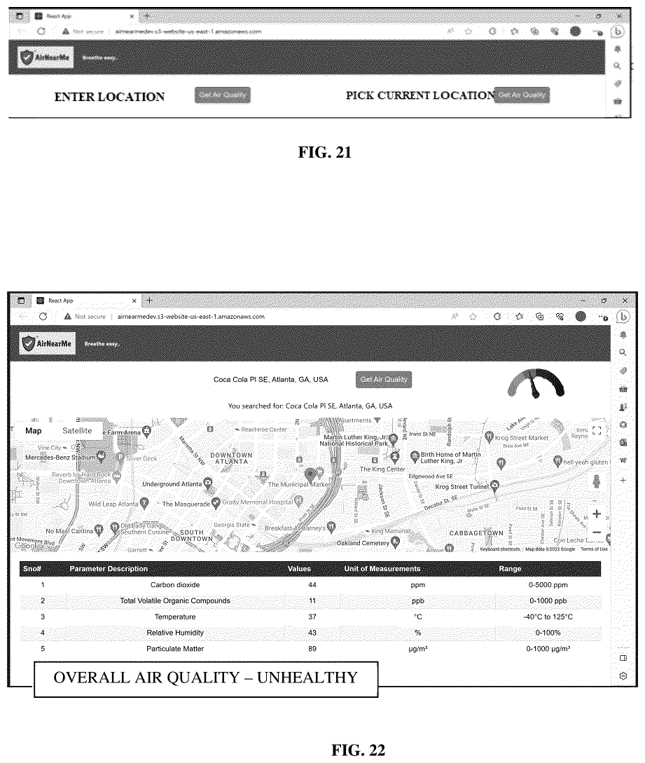

FIG. 21 shows a smartphone application for accessing Air Quality parameters according to an embodiment. The air quality can be accessed for the current location using GPS of the smartphone or for a location of interest entered via a text box or searched using the maps.

FIG. 22 shows a smartphone application displaying Air Quality parameters of a location of interest according to an embodiment. The air quality is displayed for a location which is of interest to the user. In an embodiment both qualitative and quantitative data is displayed. In an embodiment, a pictorial depiction of air quality meter, qualitative data, of the current location is displayed at the location along with the quantitative data.

The descriptions of the one or more embodiments are for purposes of illustration but are not exhaustive or limiting to the embodiments described herein. Many modifications and variations will be apparent to those of ordinary skill in the art without departing from the scope and spirit of the described embodiments. The terminology used herein best explains the principles of the embodiments, the practical application and/or technical improvement over technologies found in the marketplace, and/or to enable others of ordinary skill in the art to understand the embodiments described herein.

### INCORPORATION BY REFERENCE

All references, including granted patents and patent application publications, referred herein are incorporated herein by reference in their entirety.
U.S. patent Ser. No. 11/394,462B2 titled “Systems and methods for collecting, managing, and leveraging crowdsourced data”.
U.S. patent Ser. No. 11/112,395B2 titled “Networked air quality monitoring system”.
U.S Patent Application Publication Number US20170023509A1 titled “Crowdsourced wearable sensor system”.
U.S Patent Application Publication Number US20200139165A1, titled “Systems and methods for respiratory health management”.
U.S. Pat. No. 8,171,795B1, titled “Self-exciting, self-sensing piezoelectric cantilever sensor for detection of airborne analytes directly in air”.
U.S. Pat. No. 7,578,973B2, titled “Devices for continuous sampling of airborne particles using a regenerative surface”.

1-46. (canceled)

47. A system comprising:
a sensor module operable to detect and measure one or more air pollutants at a geographic location via one or more sensors and generate air quality data;
a cloud based application comprising a data processing module operable to receive, via a communication module, the air quality data and derive a first air quality index;
a user device comprising an application program operable to receive, via the communication module, the first air quality index; and
the application program operable to suggest an alternate location having a second air quality index, wherein the alternate location has better air quality than the geographic location; and
wherein the air quality data comprises one or more air quality parameters, the geographic location corresponding to the one or more air quality parameters and a time stamp; and
wherein the system is operable to provide real-time data of an air quality of the geographic location and the alternate location.

48. The system of claim 47, wherein the system is operable to generate the first air quality index and the second air quality index by crowdsourcing via cloud computing.

49. The system of claim 47, wherein the system is operable to generate an air quality index for a future period based on current air quality information and a history of air quality information.

50. The system of claim 47, wherein the system is operable to monitor air quality of an indoor space, wherein the indoor space comprises one or more of an office space, a house, a restaurant, a café, a movie theater, a shopping mall, an office buildings, indoor spaces of a school, indoor spaces of a university, an indoor sports facility, an inside space of a public transportation vehicle, an indoor space of a private transport vehicle, an indoor space of a hospital and healthcare facility, an underground parking lot, and an indoor concert venues.

51. The system of claim 50, wherein the system further comprises a control module operable to control a sterilization and circulation unit operable to remedy the air quality of the indoor space.

52. The system of claim 47, wherein the system is operable to monitor air quality of an outdoor space. wherein the outdoor space comprises one or more of a park, a garden, a beach, a waterfront area, a playground, a sports field, an outdoor sports stadium, a hiking trail, a nature reserves, a public square, a plaza, an open-air market, an outdoor dining area, a patio, a campground, and a rooftop terrace.

53. The system of claim 47, wherein the sensor module comprises a sensor array with a plurality of air quality sensors, comprising a gas sensor, a particle sensor, and an environmental sensor.

54. The system of claim 47, wherein the one or more air quality parameters comprise CO2, volatile organic compounds, particulate matter, pathogens, temperature, humidity, and airflow.

55. The system of claim 47, wherein the sensor module comprises a global positioning system (GPS) sensor operable to generate the geographic location comprising longitude and latitude data.

56. The system of claim 47, wherein the sensor module further comprises a power management module comprising a sleep mode to conserve battery life when the sensor module is not in use.

57. The system of claim 47, wherein the air quality data is sent to the cloud based application by a smartphone which collects the air quality data from the sensor module via a Bluetooth connection, wherein a location data from the smartphone is also sent to the cloud based application.

58. The system of claim 47, wherein the application program generates an alert and notifies a user via one or more of an audio cue, a visual cue, a tactile cue, and a text message.

59. The system of claim 47, wherein the user device comprises a display operable to display the first air quality index.

60. The system of claim 47, wherein the data processing module comprises an artificial intelligence engine comprising a machine learning algorithm for identifying and classifying the one or more air pollutants.

61. The system of claim 60, wherein the machine learning algorithm is operable to analyze the air quality data collected from the sensor module, air quality monitoring stations and satellite imagery to identify patterns, to predict a pollution level and forecast air quality in the geographic location.

62. The system of claim 47, wherein the communication module is enabled for communication using one of a wired connection and a wireless connection, wherein the communication module comprises one or more of a Wi-Fi, a Bluetooth, and a cellular connectivity for data reception and data transmission.

63. A method, comprising:
sensing one or more air pollutants via a sensor module at a geographic location;
generating air quality data;
receiving the air quality data from the sensor module by an application program on a user device via a communication module;
transmitting the air quality data to a server via the communication module;
computing an air quality index from the air quality data via a data processing module;
deriving an air quality rating corresponding to the air quality index; and
transmitting the air quality rating to the application program on the user device; and
wherein the air quality data comprises one or more air quality parameters, the geographic location corresponding to the one or more air quality parameters and a time stamp; and
wherein the method is operable to implement a crowdsourced sensor system for determining air quality.

64. The method of claim 63, wherein the method further comprises controlling an air purification system via a control module based on the air quality rating to improve the air quality.

65. The method of claim 63, wherein the method is configured for predicting the air quality rating in a region comprising:
collecting historical air quality data from one or more sources of the sensor module;
using a machine learning algorithm to identify a pattern and a trend in the historical air quality data;
generating a predictive model for air quality for a future period;
updating the predictive model with real-time air quality data;
disseminating the air quality for the future period to public; and
issuing alerts and recommendations based on a predicted air quality.

66. A non-transitory computer-readable medium having stored thereon instructions executable by a computer system to perform operations comprising:
receiving an air quality data on a server from an application program on a user device via a communication module;
computing an air quality index from the air quality data via a data processing module;
deriving an air quality rating corresponding to the air quality index; and
transmitting the air quality rating to the application program on the user device; and
wherein the air quality data comprises one or more air quality parameters, a geographic location corresponding to the one or more air quality parameters and a time stamp; and
wherein the operations are operable to implement a crowdsourced system for determining air quality.
